(19) United States
(12) Patent Application Publication
(10) Pub. No.: US 2008/0064648 A1
(43) Pub. Date: Mar. 13, 2008

(54) Methods and Compositions Relating to Lipid Accumulation

(75) Inventors: Gary Ruvkun, Newton, MA (US); Ho Yi Mak, Boston, MA (US)

Correspondence Address: CLARK & ELBING LLP, 101 FEDERAL STREET, BOSTON, MA, 02110, US

(73) Assignee: The General Hospital Corporation, Boston, MA (US)

(21) Appl. No.: 10/556,649

(22) PCT Filed: Jun. 16, 2004
(86) PCT No.: PCT/US04/19186

§ 371 Date: May 21, 2007

Related U.S. Application Data
(60) Provisional application No. 60/478,878, filed on Jun. 16, 2003.
(60) Provisional application No. 60/483,199, filed on Jun. 27, 2003.
(60) Provisional application No. 60/550,257, filed on Mar. 5, 2004.
(60) Provisional application No. 60/570,374, filed on May 12, 2004.

(51) Int. Cl. A61K 31/70; A01K 67/00; C07H 21/04; C12Q 1/20; C12Q 1/68; G01N 33/53

(52) U.S. Cl. 514/44; 435/29; 435/6; 435/7.1; 536/23.1; 536/24.5; 800/8

(57) ABSTRACT

In general, this invention relates to nucleic acid and amino acid sequences involved in lipid accumulation, screening methods for identifying genes that regulate lipid accumulation, and the use of these sequences as targets for the diagnosis, treatment, and prevention of obesity and obesity-related diseases.

### BACKGROUND OF THE INVENTION

In general, this invention relates to nucleic acid and amino acid sequences involved in lipid accumulation, screening methods for identifying genes that regulate lipid accumulation, and the use of these sequences as targets for the diagnosis, treatment, and prevention of obesity and obesity-related diseases.

Fat metabolism is controlled by a regulatory loop that exists between the central nervous system (CNS) and adipocytes. Adipocytes are specialized cells that store energy in the form of fat droplets, composed primarily of triglycerides. These fat droplets are thought to form by pinching off from membranes of the endoplasmic reticulum. Access to these fat stores is regulated by a protective protein coat, which limits their exposure to cellular lipases, enzymes which breakdown fat. Adipocytes communicate with the CNS via peptide and hormonal signals that carry information regarding the peripheral energy state. In response to these signals, the CNS controls food seeking or satiety behaviors in order to maintain energy homeostasis.

Large gaps remain in our understanding of the cell biology of fat storage, fat droplet biogenesis, and fat droplet size regulation. Moreover, the global regulators of fat metabolism, the interplay of food signals and hormones, and the genetic and environmental factors that influence body weight are still poorly understood. Addressing these deficits is crucial given the devastating impact of obesity on human health throughout the developed world. The deregulation of body weight is associated with obesity, atherosclerosis, type II diabetes mellitus, and osteoarthritis of body joints. Conservative estimates of economic costs associated with the adverse health effects of obesity range between 2% to 7% of total health costs in the developed world. In the United States, for example, diabetes, one of the diseases associated and exacerbated by obesity, is thought to affect over 16 million individuals at an annual cost of over 92 billion dollars.

As current therapies offer limited effectiveness in treating obesity and obesity-related disease, a need exists for new therapeutic targets.

### SUMMARY OF THE INVENTION

As described below, the present invention generally features nucleic acid and amino acid sequences that regulate lipid accumulation. These sequences may be used in screening methods for identifying genes that function in lipid accumulation. In addition, the invention provides for the use of these genes and their encoded proteins as therapeutic targets for the diagnosis, treatment, and prevention of lipid accumulation disorders, obesity, and obesity-related diseases.

In one aspect, the invention generally features a method for identifying a nucleic acid molecule encoding a polypeptide that regulates lipid accumulation. The method involves providing a mutagenized nematode having a pre-existing mutation in a nucleic acid molecule selected from any one or more of the group consisting of kat-1, kat-2, bbs-1, egl-4, ald-1, che-2, daf-6, osm-5, C29E6.4, tax-2, and tax-4; contacting the nematode with a dye that stains body fat; and comparing the body fat staining of the mutagenized nematode to a control nematode, where a mutation in a nucleic acid molecule encoding a polypeptide that regulates lipid accumulation is identified by an alteration in body fat staining.

In another aspect, the invention features a method for identifying a nucleic acid molecule that encodes a polypeptide that regulates lipid accumulation. The method involves contacting a nematode having a pre-existing mutation in a nucleic acid molecule selected from any one or more of the group consisting of kat-1, kat-2, bbs-1, egl-4, ald-1, che-2, daf-6, osm-5, C29E6.4, tax-2 and tax-4 with a candidate inhibitory nucleic acid; contacting the nematode with a dye that stains body fat; and comparing the body fat staining of the nematode contacted with the inhibitory nucleic acid molecule to a control nematode, where an alteration in body fat staining identifies the sense nucleic acid molecule corresponding to the inhibitory nucleic acid molecule as a nucleic acid molecule encoding a polypeptide that regulates lipid accumulation.

In another aspect, the invention features a method for identifying a candidate compound that modulates lipid accumulation. The method involves providing a cell (e.g., a mammalian cell or a nematode cell) expressing at least one nucleic acid molecule containing a mutation, where the nucleic acid molecule is selected from the group consisting of any one or more of kat-1, kat-2, egl-4, ald-1, che-2, daf-6, osm-5, C29E6.4, tax-2, and tax-4, or an ortholog thereof; contacting the cell with a candidate compound; and comparing the expression of the nucleic acid molecule in the cell contacted with the candidate compound with the expression of the nucleic acid molecule in a control cell, where an alteration in the expression identifies the candidate compound as a candidate compound that modulates lipid accumulation. In one embodiment, the cell contains at least two, three, four, five, or six nucleic acid molecules containing mutations.

In another aspect, the invention features a method for identifying a candidate compound that modulates lipid accumulation. The method involves providing a nematode cell expressing at least one nucleic acid molecule containing a mutation, where the nucleic acid molecule is selected from any one or more of the group consisting of kat-1, kat-2, egl-4, ald-1, che-2, daf-6, osm-5, C29E6.4, tax-2 and tax-4; contacting the cell with a candidate compound; and comparing the expression of the nucleic acid molecule in the cell contacted with the candidate compound with the expression of the nucleic acid molecule in a control cell, where an alteration in the expression identifies the candidate compound as a candidate compound that modulates lipid accumulation. In one embodiment, the screening method identifies a compound that increases or decreases transcription of the nucleic acid. In another embodiment, the screening method identifies a compound that increases or decreases translation of an mRNA transcribed from the nucleic acid. In one preferred embodiment, the cell is in a nematode. In another embodiment, the cell contains at least two nucleic acid molecules containing mutations.

In another aspect, the invention features a method for identifying a candidate compound that regulates lipid accumulation. The method involves providing a cell (e.g., a nematode cell or a mammalian cell) expressing a polypeptide selected from any one or more of the group consisting of KAT-1, KAT-2, EGL-4, ALD-1, CHE-2, DAF-6, OSM-5, C29E6.4, TAX-2, and TAX-4, or a fragment thereof, or an ortholog thereof; contacting the cell with a candidate compound; and comparing the biological activity of the polypeptide in the cell contacted with the candidate compound to a control cell, where an alteration in the biological activity of the polypeptide identifies the candidate compound as a candidate compound that modulates lipid accumulation. In one preferred embodiment, the nematode cell is in a nematode. In other embodiments, the biological activity is monitored with an enzymatic assay, an immunological assay, or by detecting fat levels.

In another aspect, the invention features a method for identifying a candidate compound that modulates lipid accumulation. The method involves contacting a polypeptide selected from any one or more of the group consisting of KAT-1, KAT-2, EGL-4, ALD-1, CHE-2, DAF-6, OSM-5, C29E6.4, TAX-2, or TAX-4, or a fragment thereof, or an ortholog thereof, with a candidate compound; and detecting binding of the candidate compound to the polypeptide, where the binding identifies the candidate compound as a compound that modulates lipid accumulation.

In another aspect, the invention features a transgenic organism (e.g., a nematode or a rat) overexpressing a kat-1, kat-2, egl-4 gain- of-function, bbs-1, C29E6.4, or ald-1 nucleic acid molecule, or a fragment thereof, where expression of the protein product encoded by the nucleic acid molecule alters lipid accumulation in the organism. In various embodiments, when the organism is a mammal (e.g., a mouse or rat) then the nucleic acid molecule is an ortholog of a KAT-1, KAT-2, C29E6.4, or EGL-4 gain-of-function nucleic acid molecule.

In another aspect, the invention features a nematode containing a mutation in a nucleic acid sequence selected from any one or more of the group consisting of kat-1, kat-2, egl-4 gain-of-function, ald-1, C29E6.4, and bbs-1.

In another aspect, the invention features a mammal containing at least one mutation in a nucleic acid sequence selected from any one or more of the group consisting of mammalian orthologs of kat-1, che-2, daf-6, C29E6.4, osm-5, tax-2, tax-4 or egl-4 gain-of-function.

In another aspect, the invention features a double-stranded RNA corresponding to at least a portion of a nucleic acid molecule selected from any one or more of the group consisting of kat-1, kat-2, egl-4, ald-1, che-2, daf-6, C29E6.4, osm-5, tax-2, and tax-4, or an orthog thereof, where the double-stranded RNA when introduced to a cell is capable of altering the level of lipid accumulation in the cell.

In another aspect, the invention features an antisense nucleic acid molecule complementary to at least a portion of a nucleic acid molecule selected from any one or more of the group consisting of kat-1, kat-2, egl-4, ald-1, che-2, daf-6, C29E6.4, osm-5, tax-2, and tax-4, or an ortholog thereof, where the antisense nucleic acid molecule when introduced to a cell is capable of decreasing expression from the nucleic acid molecule to which it is complementary.

In another aspect, the invention features a method for diagnosing an organism (e.g., a mammal, such as a human) having, or having a propensity to develop a lipid accumulation disorder, obesity, or an obesity-related disease. The method involves detecting an alteration in the sequence of a nucleic acid molecule selected from any one or more of the group consisting of kat-1, kat-2, egl-4, ald-1, che-2, daf-6, C29E6.4, osm-5, tax-2, and tax-4, or an ortholog thereof.

In a related aspect, the invention features a method for diagnosing an organism (e.g., a mammal, such as a human) having, or having a propensity to develop, a lipid accumulation disorder, obesity, or an obesity-related disease. The method involves detecting an alteration in the expression of a nucleic acid molecule selected from any one or more of the group consisting of kat-1, kat-2, egl-4, ald-1, che-2, daf-6, osm-5, C29E6.4, tax-2, and tax-4, or an ortholog thereof, relative to the wild-type level of expression.

In a related aspect, the invention features a method for diagnosing an organism (e.g., a mammal, such as a human) having, or having a propensity to develop, a lipid accumulation disorder, obesity, or an obesity-related disease. The method involves detecting an alteration in the biological activity of a polypeptide selected from any one or more of the group consisting of kat-1, kat-2, egl-4, ald-1, che-2, daf-6, osm-5, C29E6.4, tax-2, and tax-4, or an ortholog thereof, relative to the wild-type level of activity.

In another aspect, the invention features a method for modulating lipid accumulation in an organism (e.g., a mammal, such as a human). The method involves contacting the organism with an antisense nucleic acid molecule that complements a portion of a nucleic acid molecule selected from any one or more of the group consisting of kat-1, kat-2, egl-4, ald-1, che-2, daf-6, osm-5, C29E6.4, tax-2 and tax-4, or an ortholog thereof.

In another aspect, the invention features a method for modulating lipid accumulation in an organism (e.g., a mammal, such as a human). The method involves contacting the organism with a dsRNA nucleic acid molecule that corresponds to at least a portion of a nucleic acid molecule selected from any one or more of the group consisting of kat-1, kat-2, egl-4, ald-1, che-2, daf-6, osm-5, C29E6.4, tax-2 and tax-4, or an ortholog thereof.

In another aspect, the invention features a method for modulating lipid accumulation in an organism. The method involves contacting the organism with a nucleic acid molecule selected from the group consisting of kat-1, kat-2, egl-4, ald-1, che-2, daf-6, osm-5, C29E6.4, tax-2, and tax-4, or an ortholog thereof.

In another aspect, the invention features a pharmaceutical composition containing a polypeptide, or portion thereof, selected from any one or more of the group consisting of mammalian orthologs of KAT-1, KAT-2, EGL-4, ALD-1, CHE-2, DAF-6, OSM-5, C29E6.4, TAX-2, AND TAX-4.

In a related aspect, the invention features a pharmaceutical composition containing a nucleic acid molecule or portion thereof, selected from any one or more of the group consisting of mammalian orthologs of kat-1, kat-2, egl-4, ald-1, che-2, daf-6, osm-5, C29E6.4, tax-2, and tax-4.

In various embodiments of the above aspects, a nucleic acid molecule is a kat-1, kat-2, egl-4, egl-4 gain-of-function, ald-1, bbs-1, che-2, daf-6, osm-5, C29E6.4, tax-2, and tax-4, or a fragment thereof, or an ortholog thereof. In preferred embodiments of any of the above aspects, the ortholog is a mammalian ortholog.

By “lipid accumulation” is meant an increase in the level of at least one form of fatty acid in a cell, tissue, or organ of an animal. Lipid accumulation is detected, for example, by Nile red staining, by C1-BODIPY-C12 staining, by lipid extraction and gas chromatography/mass spectroscopy, or by any other method known in the art. Lipid accumulation refers to, for example, an increase in the level of total lipids or an increase in at least one type of fatty acid (e.g., very long chain fatty acids).

By “KAT-1 polypeptide” is meant a protein, or fragment thereof, having at least 27% identity or 48% similarity to the C. elegans KAT-1 (T02G5.8) polypeptide, having peroxisomal 3-ketoacyl-coA thiolase activity, containing a CSSGL motif, and/or functioning in lipid accumulation. Exemplary KAT-1 polypeptides include the C. elegans polypeptide encoded by T02G5.8 and the human polypeptide ACAA1 (GenBank Accession No: NP_{—}001598), and orthologs thereof.

By “kat-1 nucleic acid molecule” is meant a nucleic acid molecule that encodes a KAT-1 polypeptide.

By “KAT-2 polypeptide” is meant a protein, or fragment thereof, having peroxisomal 3-ketoacyl-coA thiolase activity, having at least 57% identity or 75% similarity to Y57A10C.6 or human SCPx (Accession no. P22307), and/or functioning in lipid accumulation.

By “kat-2 nucleic acid molecule” is meant a nucleic acid molecule that encodes a KAT-2 polypeptide.

By “BBS-1 polypeptide” is meant a protein, or fragment thereof, having at least 27% identity or 75% similarity to C. elegans bbs-1 (Y105E8A.5) and/or functioning in lipid accumulation. Inactivation, of a BBS-1 polypeptide in combination with a kat-1 loss-of-function mutation results in a kat-1 dependent increase in lipid accumulation. Human BBS-1 (Accession no. Q8NFJ9) is 27% identical to the C. elegans BBS-1 polypeptide.

By “bbs-1 nucleic acid molecule” is meant a nucleic acid molecule that encodes a BBS-1 polypeptide

By “EGL-4 polypeptide” is meant a protein, or fragment thereof, having at least 46% identity or 63% similarity to C. elegans EGL-4, having cGMP dependent protein kinase activity, and/or functioning in lipid accumulation. Ectopic activation of an EGL-4 polypeptide in combination with a kat-1 loss-of-function mutation results in a kat-1 dependent increase in lipid accumulation. Exemplary EGL-4 polypeptides include F55A8.2a, 2b, 2c, 2d and human cGK1-beta (Genbank Accession No. P14619).

By “egl-4 nucleic acid molecule” is meant a nucleic acid molecule that encodes an EGL-4 polypeptide.

By “ALD-1 polypeptide” is meant a protein, or fragment thereof, having at least 57% identity or 72% similarity to C. elegans ALD-1 (this protein is now called PMP-4 ) (T02D1.5), having very long chain fatty acids transporter activity, and/or functioning in lipid accumulation. Human ALDP (Genbank Accession no. P33897) is an exemplary ALD-1 polypeptide.

By “ald-1 nucleic acid molecule” is meant a nucleic acid molecule that encodes an ALD-1 polypeptide.

By “CHE-2 polypeptide” is meant a protein, or fragment thereof, having at least 41% identity or 65% similarity to C. elegans CHE-2 (F38G1.1), functioning in ciliated neuron maintenance, and/or functioning in lipid accumulation. Inactivation of a CHE-2 polypeptide in combination with a kat-1 loss-of-function mutation results in a kat-1 increase in lipid accumulation. Exemplary CHE-2 polypeptides include Genbank Accession No. Q9P2H3.

By “che-2 nucleic acid molecule” is meant a nucleic acid molecule that encodes a CHE-2 polypeptide.

By “DAF-6 polypeptide” is meant a protein, or fragment thereof, having at least 20% identity or 36% similarity to C. elegans DAF-6 (F31F6.5) and/or functioning in lipid accumulation. Inactivation of a DAF-6 polypeptide in combination with a kat-1 loss-of-function mutation results in a kat-1 dependent increase in lipid accumulation. Exemplary DAF-6 polypeptides include human patched-2 (Genbank Accession No. Q9Y6C5).

By “daf-6 nucleic acid molecule” is meant a nucleic acid molecule that encodes a DAF-6 polypeptide.

By “TAX-2 polypeptide” is meant a protein subunit of a cyclic nucleotide-gated channel, or a fragment thereof, that functions in lipid accumulation and/or that has at least 33% identity or 51% similarity to C. elegans TAX-2 (F36F2.5). One exemplary DAF-6 polypeptide is human cGMP gated cation channel beta subunit (Accession number Q9UMG2). Inactivation of a TAX-2 polypeptide in combination with a kat-1 loss-of-function mutation results in a kat-1 dependent increase in lipid accumulation.

By “tax-2 nucleic acid molecule” is meant a nucleic acid molecule that encodes a TAX-2 polypeptide.

By “TAX-4 polypeptide” is meant a protein subunit of a cyclic nucleotide-gated channel, or a fragment thereof, that functions in lipid accumulation and/or that has at least 48% identity or 61% similarity to C. elegans TAX-4 (ZC84.2). Inactivation of a TAX-4 polypeptide in combination with a kat-1 loss-of-function mutation results in a kat-1 dependent increase in lipid accumulation. One exemplary TAX-4 polypeptide is human cGMP-gated cation channel alpha subunit (Genbank Accession No: P29973).

By “tax-4. nucleic acid molecule” is meant a nucleic acid molecule that encodes a TAX-4 polypeptide.

By “TUB-1 polypeptide” is meant a protein, or fragment thereof, having at least 60% identity or 75% similarity to the carboxy terminal half of a protein encoded by tub-1 (F10B5.4), which functions in lipid accumulation.

By “tub-1 nucleic acid molecule” is meant a nucleic acid molecule that encodes a TUB-1 polypeptide.

By “OSM-5 polypeptide” is meant a protein, or fragment thereof, having at least 44% identity or 64% similarity C. elegans OSM-5 (Y41G9A.1), functioning in the maintenance of a ciliated neuron, and/or functioning in lipid accumulation. Inactivation of an OSM-5 polypeptide in combination with a kat-1 loss-of-function mutation results in a kat-1 dependent increase in lipid accumulation.

By “osm-5 nucleic acid molecule” is meant a nucleic acid molecule that encodes an OSM-5 polypeptide.

By a “C29E6.4 polypeptide” is meant a protein that has at least 85% identity to a C29E6.4 and contains at least one PAN domain, and/or that functions in lipid accumulation. A loss-of-function mutation in a C29E6.4 polypeptide enhances lipid accumulation in a kat-1(−) background.

By a “C29E6.4 nucleic acid molecule” is meant a nucleic acid molecule that encodes a C29E6.4 polypeptide.

By “an alteration” is meant an increase or decrease. The alteration may be by as little as 5%, 10%, or 15%, more preferably by at least 25%, 30%, 40%, 50%, or 60%, 70%, or even by as much as 80%, 90%, 100%, 150%, 200%, 300%, or more.

By “isolated nucleic acid” is meant a polynucleotide (e.g., a DNA) that is free of the genes which, in the naturally-occurring genome of the organism from which the nucleic acid molecule of the invention is derived, flank the gene. The term therefore includes, for example, a recombinant DNA that is incorporated into a vector; into an autonomously replicating plasmid or virus; or into the genomic DNA of a prokaryote or eukaryote; or that exists as a separate molecule (for example, a cDNA or a genomic or cDNA fragment produced by PCR or restriction endonuclease digestion) independent of other sequences. In addition, the term includes an RNA molecule that is transcribed from a DNA molecule, as well as a recombinant DNA that is part of a hybrid gene encoding additional polypeptide sequence.

By “polypeptide” is meant any chain of amino acids, regardless of length or post-translational modification (for example, glycosylation or phosphorylation).

By an “isolated polypeptide” is meant a polypeptide of the invention that has been separated from components that naturally accompany it. Typically, the polypeptide is isolated when it is at least 60%, by weight, free from the proteins and naturally-occurring organic molecules with which it is naturally associated. Preferably, the preparation is at least 75%, more preferably at least 90%, and most preferably at least 99%, by weight, a polypeptide of the invention. An isolated polypeptide of the invention may be obtained, for example, by extraction from a natural source, by expression of a recombinant nucleic acid encoding such a polypeptide; or by chemically synthesizing the protein. Purity can be measured by any appropriate method, for example, column chromatography, polyacrylamide gel electrophoresis, or by HPLC analysis.

By “substantially identical” is meant a polypeptide or nucleic acid molecule exhibiting at least 50% identity to a reference amino acid sequence (for example, any one of the amino acid sequences described herein) or nucleic acid sequence (for example, any one of the nucleic acid sequences described herein). Preferably, such a sequence is at least 60%, more preferably 80%, and most preferably 90% or even 95% identical at the amino acid level or nucleic acid to the sequence used for comparison.

Sequence identity is typically measured using sequence analysis software (for example, Sequence Analysis Software Package of the Genetics Computer Group, University of Wisconsin Biotechnology Center, 1710 University Avenue, Madison, Wis. 53705, BLAST, BESTFIT, GAP, or PILEUP/PRETTYBOX programs). Such software matches identical or similar sequences by assigning degrees of homology to various substitutions, deletions, and/or other modifications. Conservative substitutions typically include substitutions within the following groups: glycine, alanine; valine, isoleucine, leucine; aspartic acid, glutamic acid, asparagine, glutamine; serine, threonine; lysine, arginine; and phenylalanine, tyrosine. In an exemplary approach to determining the degree of identity, a BLAST program may be used, with a probability score between e⁻³ and e⁻¹⁰⁰ indicating a closely related sequence.

By “transformed cell” is meant a cell into which (or into an ancestor of which) has been introduced, by means of recombinant DNA techniques, a polynucleotide molecule encoding (as used herein) a polypeptide of the invention.

By “positioned for expression” is meant that the polynucleotide of the invention (e.g., a DNA molecule) is positioned adjacent to a DNA sequence which directs transcription and translation of the sequence (i.e., facilitates the production of, for example, a recombinant polypeptide of the invention, or an RNA molecule).

By “purified antibody” is meant an antibody which is at least 60%, by weight, free from proteins and naturally-occurring organic molecules with which it is naturally associated. Preferably, the preparation is at least 75%, more preferably 90%, and most preferably at least 99%, by weight, antibody. A purified antibody of the invention may be obtained, for example, by affinity chromatography using a recombinantly-produced polypeptide of the invention and standard techniques.

By “specifically binds” is meant a compound or antibody which recognizes and binds a polypeptide of the invention but which does not substantially recognize and bind other molecules in a sample, for example, a biological sample, which naturally includes a polypeptide of the invention.

By “derived from” is meant isolated from or having the sequence of a naturally-occurring sequence (e.g., a cDNA, genomic DNA, synthetic, or combination thereof).

By “immunological assay” is meant an assay that relies on an immunological reaction, for example, antibody binding to an antigen. Examples of immunological assays include ELISAs, Western blots, immunoprecipitations, and other assays known to the skilled artisan.

By “inhibitory nucleic acid” is meant a nucleic acid that reduces or eliminates expression or biological activity of a gene or protein of interest. “Inhibitory nucleic acids” include, without limitation, antisense nucleic acids, double stranded RNAs (dsRNA), or small interfering RNAs (siRNA), or analogs thereof.

By “anti-sense” is meant a nucleic acid, or analog thereof, regardless of length, that is complementary to the coding strand or mRNA of a nucleic acid sequence. In one embodiment, an antisense RNA is introduced to an individual cell, tissue, organ, or to a whole animals. Desirably the anti-sense nucleic acid is capable of decreasing the expression or biological activity of a nucleic acid or amino acid sequence. In one embodiment, the decrease in expression or biological activity is at least 10%, relative to a control, more desirably 25%, and most desirably 50%, 60%, 70%, 80%, 90%, or more. The anti-sense nucleic acid may contain a modified backbone, for example, phosphorothioate, phosphorodithioate, or other modified backbones known in the art, or may contain non-natural internucleoside linkages.

By “double stranded RNA” is meant a complementary pair of sense and antisense RNAs regardless of length. In one embodiment, these dsRNAs are introduced to an individual cell, tissue, organ, or to a whole animals. For example, they may be introduced systemically via the bloodstream. Desirably, the double stranded RNA is capable of decreasing the expression or biological activity of a nucleic acid or amino acid sequence. In one embodiment, the decrease in expression or biological activity is at least 10%, relative to a control, more desirably 25%, and most desirably 50%, 60%, 70%, 80%, 90%, or more. The anti-sense nucleic acid may contain a modified backbone, for example, phosphorothioate, phosphorodithioate, or other modified backbones known in the art, or may contain non-natural internucleoside linkages.

By “siRNA” is meant a double stranded RNA that complements a region of an mRNA. Optimally, an siRNA is 22-24 nucleotides in length and has a 2 base overhang at its 3′ end. These dsRNAs can be introduced to an individual cell or to a whole animal, for example, they may be introduced systemically via the bloodstream. Such siRNAs are used to downregulate mRNA levels or promoter activity. In one embodiment, the decrease in expression or biological activity is at least 10%, relative to a control, more desirably 25%, and most desirably 50%, 60%, 70%, 80%, 90%, or more. The siRNA may contain a modified backbone, for example, phosphorothioate, phosphorodithioate, or other modified backbones known in the art, or may contain non-natural internucleoside linkages.

By “hybridize” is meant pair to form a double-stranded molecule between complementary polynucleotide sequences, or portions thereof, under various conditions of stringency. (See, e.g., Wahl, G. M. and S. L. Berger (1987) Methods Enzymol. 152:399; Kimmel, A. R. (1987) Methods Enzymol. 152:507) For example, stringent salt concentration will ordinarily be less than about 750 mM NaCl and 75 mM trisodium citrate, preferably less than about 500 mM NaCl and 50 mM trisodium citrate, and most preferably less than about 250 mM NaCl and 25 mM trisodium citrate. Low stringency hybridization can be obtained in the absence of organic solvent, e.g., formamide, while high stringency hybridization can be obtained in the presence of at least about 35% formamide, and most preferably at least about 50% formamide. Stringent temperature conditions will ordinarily include temperatures of at least about 30° C., more preferably of at least about 37° C., and most preferably of at least about 42° C. Varying additional parameters, such as hybridization time, the concentration of detergent, e.g., sodium dodecyl sulfate (SDS), and the inclusion or exclusion of carrier DNA, are well known to those skilled in the art. Various levels of stringency are accomplished by combining these various conditions as needed. In a preferred embodiment, hybridization will occur at 30° C. in 750 mM NaCl, 75 mM trisodium citrate, and 1% SDS. In a more preferred embodiment, hybridization will occur at 37° C. in 500 mM NaCl, 50 mM trisodium citrate, 1% SDS, 35% formamide, and 100 μg/ml denatured salmon sperm DNA (ssDNA). In a most preferred embodiment, hybridization will occur at 42° C. in 250 mM NaCl, 25 mM trisodium citrate, 1% SDS, 50% formamide, and 200 μg/ml ssDNA. Useful variations on these conditions will be readily apparent to those skilled in the art.

For most applications, washing steps that follow hybridization will also vary in stringency. Wash stringency conditions can be defined by salt concentration and by temperature. As above, wash stringency can be increased by decreasing salt concentration or by increasing temperature. For example, stringent salt concentration for the wash steps will preferably be less than about 30 mM NaCl and 3 mM trisodium citrate, and most preferably less than about 15 mM NaCl and 1.5 mM trisodium citrate. Stringent temperature conditions for the wash steps will ordinarily include a temperature of at least about 25° C., more preferably of at least about 42° C., and most preferably of at least about 68° C. In a preferred embodiment, wash steps will occur at 25° C. in 30 mM NaCl, 3 mM trisodium citrate, and 0.1% SDS. In a more preferred embodiment, wash steps will occur at 42° C. in 15 mM NaCl, 1.5 mM trisodium citrate, and 0.1% SDS. In a most preferred embodiment, wash steps will occur at 68° C. in 15 mM NaCl, 1.5 mM trisodium citrate, and 0.1% SDS. Additional variations on these conditions will be readily apparent to those skilled in the art. Hybridization techniques are well known to those skilled in the art and are described, for example, in Benton and Davis (Science 196:180, 1977); Grunstein and Hogness (Proc. Natl. Acad. Sci., USA 72:3961, 1975); Ausubel et al. (Current Protocols in Molecular Biology, Wiley Interscience, New York, 2001); Berger and Kimmel (Guide to Molecular Cloning Techniques, 1987, Academic Press, New York); and Sambrook et al., Molecular Cloning: A Laboratory Manual, Cold Spring Harbor Laboratory Press, New York.

By “ortholog” is meant any polypeptide or nucleic acid molecule of an organism that is highly related to a reference protein or nucleic acid sequence from another organism. The degree of relatedness may be expressed as the probability that a reference protein would identify a sequence, for example, in a blast search. The probability that a reference sequence would identify a random sequence as an ortholog is extremely low, less than e⁻¹⁰, e⁻²⁰, e⁻³⁰, e⁻⁴⁰, e⁻⁵⁰, e⁻⁷⁵, e⁻¹⁰⁰. The skilled artisan understands that an ortholog is likely to be functionally related to the reference protein or nucleic acid sequence. In other words, the ortholog and its reference molecule would be expected to fulfill similar, if not equivalent, functional roles in their respective organisms. For example, a C. elegans lipase and its mammalian ortholog would both be expected to fulfill the enzymatic function of lipases in their respective organisms. It is not required that an ortholog, when aligned with a reference sequence, have a particular degree of amino acid sequence identity to the reference sequence. A protein ortholog might share significant amino acid sequence identity over the entire length of the protein, for example, or, alternatively, might share significant amino acid sequence identity over only a single functionally important domain of the protein. Orthologs may be identified using methods provided herein. The functional role of an ortholog may be assayed using methods well known to the skilled artisan, and described herein. For example, function might be assayed in vivo or in vitro using a biochemical, immunological, or enzymatic assays; transformation rescue, Nile Red or BODIPY assays for the effect of gene inactivation on fat content, storage, or mobilization; such fat content assays, as described herein, may be carried out in a whole animal (e.g., C. elegans) or in tissue culture; function may also be assayed by gene inactivation (e.g., by RNAi, siRNA, or gene knockout), or gene over-expression, as well as by other methods.

By “fat metabolism” is meant, for example, fat storage, fat deposition, fat breakdown, fat droplet biogenesis, fat mobilization, or the increase, decrease, or maintenance of the fat content of an organism.

By “transgene” is meant any piece of DNA which is inserted by artifice into a cell and typically becomes part of the genome of the organism which develops from that cell, or is maintained as a heritable array. Such a transgene may include a gene that is partly or entirely heterologous (i.e., foreign) to the transgenic organism, or may represent a gene homologous to an endogenous gene of the organism.

By “transgenic” is meant any cell that includes a DNA sequence which is inserted by artifice into a cell and becomes part of the genome of the organism which develops from that cell or is maintained as a heritable extrachromasomal array. As used herein, the transgenic organisms are generally transgenic C. elegans or transgenic vertebrates, such as, zebrafish, mice, and rats.

“Cell” as used herein may be a single-cellular organism, a cell from a multi-cellular organism, or it may be a cell contained in a multi-cellular organism.

“Differentially expressed” means a difference in the expression level of a nucleic acid. This difference may be either an increase or a decrease in expression, when compared to control conditions.

“Microarray” means a collection of nucleic acids or polypeptides from one or more organisms arranged on a solid support (for example, a chip, plate, or bead). These nucleic acids or polypeptides may be arranged in a grid where the location of each nucleic acid or polypeptide remains fixed to aid in identification of the individual nucleic acids or polypeptides. A microarray may include, for example, nucleic acids representing all, or a subset, of the open reading frames of an organism, or of the polypeptides that those open reading frames encode.

“Therapeutic compound” means a substance that has the potential of affecting the function of an organism. Such a compound may be, for example, a naturally occurring, semi-synthetic, or synthetic agent. For example, the test compound may be a drug that targets a specific function of an organism. A test compound may also be an antibiotic or a nutrient. A therapeutic compound may decrease, suppress, attenuate, diminish, arrest, or stabilize the development or progression of disease, disorder, or infection in a eukaryotic host organism.

The invention provides a number of targets that are useful for the development of drugs to treat obesity, the deregulation of fat metabolism, or a lipid accumulation disorder. In addition, the methods of the invention provide a facile means to identify therapies that are safe for use in eukaryotic host organisms (i.e., compounds which do not adversely affect the normal development, physiology, or fertility of the organism). In addition, the methods of the invention provide a route for analyzing virtually any number of compounds for effects on fat metabolism with high-volume throughput, high sensitivity, and low complexity. The methods are also relatively inexpensive to perform and enable the analysis of small quantities of active substances found in either purified or crude extract form.

Other features and advantages of the invention will be apparent from the detailed description, and from the claims.

### BRIEF DESCRIPTION OF THE DRAWINGS

FIGS. 1A and 1B shows the characterization of tub-1. FIG. 1A is a schematic diagram showing the genomic organization of tub-1; exons are depicted in black and the 3′ untranslated region is in grey. The deletions nr2004 and nr2044 are marked. FIG. 1B is a series of confocal images of an L1 larva expressing TUB-1::GFP in ciliated neurons. TUB-1::GFP was expressed most strongly in the amphid and phasmid sensory neurons. TUB-1::GFP was present in neuronal cell bodies, axons, dendrites and ciliated endings, but was excluded from the nucleus.

FIGS. 2A-2I show a synergistic increase in lipid accumulation in kat-1 tub-1 mutant worms. FIGS. 2A-2D are photomicrographs showing Nile red staining in 2-day old adult wild-type, kat-1 (mg368), tub-1 (nr2004) and kat-1(mg368) tub-1(nr2004) worms. Pictures were taken at the same exposure with a 16× objective capturing the anterior half of the worms. A Nomarski image of each worm is shown in the inset of each panel. FIGS. 2E-2H are photomicrographs showing C1-BODIPY-C12 staining of 2-day old adult wild-type kat-1(mg368), tub-1(nr2004) and kat-1(mg368) tub-1(nr2004) worms. FIG. 2I is a graph that quantifies the Nile red fluorescence of 2-day and 6-day old adult worms. The level of fluorescence of wild-type worms is set as 1 and the fold change in fluorescence in mutant worms is shown. At least two independent experiments were performed and error bars indicate the standard deviation. The number of 2-day old adult worms examined is as follows: wild-type N=30, kat-1(mg368) N=30, tub-1(nr2004) N=33, kat-1(mg368) tub-1(nr2004) N=36. The number of 6-day old adult worms examined is as follows: wild-type N=40, kat-1(mg368) N=31, tub-1(nr2004) N=35, kat-1(mg368) tub-1(nr2004) N=31.

FIG. 3A shows a peroxisomal beta-oxidation pathway.

FIG. 3B shows a peroxisomal beta-oxidation pathway, and indicates enzymes in which mutations have been identified in various species. Drosophila Bubblegum mutants have a shortened lifespan and retinal degeneration. Demyelination of central and/or peripheral nervous system occurs in patients with pseudoneonatal adrenoleukodystrophy.

FIG. 4 shows a sequence alignment of the C. elegans KAT-1 N-terminal region with homologues from other species. The asterisk indicates residues that form the catalytic site of the enzyme. The position of missense mutations is identified as are the DNA base change and allele names (e.g. mg368).

FIG. 5 shows a sequence alignment of the C. elegans KAT-1 C-terminal region with homologues from other species. The asterisk indicates residues that form the catalytic site of the enzyme. Missense mutations, DNA base changes, and allele names are indicated (e.g. mg368).

FIGS. 6A-6D are photomicrographs of worms expressing the ges-1p::GFP-PTS1KAT-1 transgene (mgEx690). The worms were allowed to lay eggs for 6 hours on plates seeded with bacteria carrying the L4440 vector control or the plasmid that directs expression of prx-5 double-stranded RNA (kindly provided by J. Ahringer). A fraction of F1 progeny subjected to prx-5 RNAi displayed retardation of larval development. Young adults were selected for confocal microscopy. The images were taken with a 63× objective and are projections of 16 optical slices (8 μm depth).

FIGS. 7A-7F show the expression pattern and focus of action of KAT-1, a peroxisomal 3-ketoacyl-coA thiolase. FIG. 7A is schematic representation of the KAT-1 protein structure showing the relative position of mutations and the corresponding changes in amino acid sequence. FIGS. 7B-7E are photomicrographs showing kat-1p::GFP expression. FIG. 7B shows expression of kat-1p::GFP in the intestine of a 3-fold stage embryo. FIG. 7C shows expression of kat-1p::GFP in the intestine of an L1 larvae. FIG. 7D shows expression of kat-1p::GFP in the intestine, pharynx, and body wall muscle of an L4 larvae. FIG. 7E shows tissue specific rescue of kat-1 in kat-1(mg368) tub-1(nr2004) worms. kat-1 cDNA was driven by kat-1, ges-1 or tub-1 promoter and the level of Nile red fluorescence in 2-day old adult worms of two independent transgenic lines was quantified. The level of fluorescence of kat-1(mg368) tub-1(nr2004) worms is set at 100% and the percent change in fluorescence in transgenic worms is shown. At least two independent experiments were performed and error bars indicate the standard error of the mean. The number of worms examined is as follows: kat-1 tub-1 N=30, mgEx681 N=31, mgEx682 N=31, mgEx683 N=28, mgEx684 N=28, mgEx685 N=29, mgEx686 N=28. FIG. 7F shows a sequence alignment of KAT-1 and its orthologues, human ACAA1 and yeast Pot1p. The conserved catalytic residues (GHP) in each of the orthologs are marked with an asterisk.

FIGS. 8A-8D are photomicrographs showing Nile red staining in wild-type, kat-1, tub-1, and kat-1 tub-1 2-day-old reproductive adult worms. Pictures were taken at the same exposure time to illustrate differences in the intensity of staining. The insets are photomicrographs of each nematode visualized using Nomarski optics.

FIG. 9 is a graph showing the quantitation of Nile red staining. For each strain, the fluorescence of 15 or more worms was measured.

FIGS. 10A-10D are photomicrographs showing Nile red staining in wild-type, kat-1, tub-1 and kat-1 tub-1 worms, respectively, as 6 day old post reproductive adult. Pictures were taken at the same exposure time to illustrate the difference in intensity of staining. The insets are photomicrographs of each nematode visualized using Nomarski optics.

FIG. 11 is a graph showing the quantitation of Nile red staining in 6-day-old adult worms. For each strain, the fluorescence of 13 or more worms was measured.

FIGS. 12A-12L are photomicrographs showing normal peroxisomal and mitochondrial morphology in kat-1 tub-1 mutant and wild-type worms. Confocal images of the anterior part of the intestine were taken with a 63× objective. FIGS. 12A-12F show wild-type 2-day old adult worms. FIG. 12C and 12I show peroxisomes in the intestine visualized by GFP that was targeted to the peroxisomal matrix (mgIs43). FIGS. 12F and 12L show mitochondria in the intestine visualized by GFP that was targeted to the mitochondrial matrix (mgIs48).

FIGS. 13A-13F show the cloning and characterization of bbs-1. FIG. 13A is a schematic representation of the BBS-1 protein structure showing the position of the mutant allele mg409. FIG. 13B is a graph showing the quantification of Nile red fluorescence in 2-day and 6-day old adult worms. The level of fluorescence in wild-type worms is normalized to 1 and the fold change in fluorescence in mutant worms is shown. At least two independent experiments were performed and error bars indicate standard deviation. The number of 2-day old adult worms examined is as follows: wild-type N=34, kat-1(mg368) N=30, bbs-1(mg409) N=32, bbs-1(mg409); kat-1(mg368) N=36, bbs-1(mg409); tub-1(nr2004) N=35. The number of 6-day old adult worms examined is as follows: wild-type N=35, kat-1(mg368) N=31, bbs-1(mg409) N=33, bbs-1(mg409); kat-1(mg368) N=31, bbs-1(mg409); tub-1(nr2004) N=24. FIG. 13C is a graph that depicts the quantification of Nile red fluorescence in 6-day old adult worms. The level of fluorescence in wild-type worms is normalized to 1 and the fold change in fluorescence in mutant worms is depicted. Two independent experiments were performed and the error bars indicate the standard deviation. The number of worms examined is as follows: wild-type N=34, bbs-1(mg409); kat-1(mg368) N=25, bbs-1(mg409); kat-1(mg368); mgEx687 N=26, kat-1(mg368); mgEx688 N=25. FIG. 13D is a series of photographs of worm culture plates that illustrate the wild-type phenotype of N2 worms, the dwelling phenotype of bbs-1(mg409); kat-1(mg368) mutant worms, and the rescue phenotype of the double mutant worms expressing transgene, mgEx687, which restored wild-type bbs-1 activity. Pictures were taken 20 hours after a single 1-day old adult worm was transferred onto a plate with abundant food and incubated at 20° C. FIG. 13E is a pair of photomicrographs that show the localization of BBS-1::GFP to the transition zone of a phasmid neuron. The left panel shows the dendrite and cilium visualized with DiI staining, and the right panel shows the same worm visualized by Nomarski optics. FIG. 13F shows the alignment of C. elegans BBS-1 and its human counterpart.

FIGS. 14A-D show that amphid sensory neurons play an important role in lipid homeostasis. FIGS. 14A is a graph showing the quantification of Nile red fluorescence in 2-day old adult worms. The level of fluorescence of wild-type worms is normalized to 1 and the fold change in fluorescence in mutant worms is shown. Two independent experiments were performed except for che-2(e1033) and error bars indicate standard deviation. This graph indicates that defects in sensory cilia or their support cells cause a synergistic increase in lipid accumulation in kat-1 mutant worms. The number of worms examined is as follows: wild-type N=32, kat-1(mg368) N=30, che-2(e1033) N=10, kat-1(mg368); che-2(e1033) N=31, osm-5(p813) N=27, kat-1(mg368); osm-5(p813) N=33, daf-6(e1377) N=36, kat-1(mg368); daf-6(e1377) N=29. FIGS. 14B, 14C, and 14D are schematic diagrams showing models for the transmission of a neuroendocrine signal from ciliated neurons to peripheral tissues that control fatty acid β-oxidation in peroxisomes and mitochondria.

FIGS. 15A-15D are photomicrographs of 6-day-old adult C. elegans showing C1-BODIPY-C12 staining of wild-type, kat-1 tub-1, che-2, and kat-1; che-2 worms.

FIGS. 16A-16C depict the characterization of the egl-4(mg410) molecular lesion. FIG. 16A is a schematic diagram showing the exon intron structure of F55A8.2a, F55A8.2b, F55A8.2c, and F55A8.2d. FIG. 16B shows the nucleic acid and amino acid sequences in the region of the mutation. FIG. 16C is a gel showing the results of an Ssp1 restriction digest of PCR amplified genomic DNA, extracted from wild-type and mg410 worms, flanking the mg410 mutation. The mg410 mutation creates an additional SspI restriction site.

FIG. 17A is a schematic diagram showing EGL-4 functional domains. FIG. 17B shows the amino acid sequence of the consensus sequence for the kinase substrate motif and the pseudo-substrate motif in. FIG. 17C shows the amino acid sequence of the pseudo-substrate motif in egl-4 loss of function and gain-of-function, mg410, mutants.

FIG. 18 is a schematic diagram that depicts the EGL-4 signaling pathway in C. elegans.

FIG. 19 is an alignment of C. elegans EGL-4 and its human ortholog. Identical amino acid residues are indicated with black shading and conservative substitutions are indicated with gray shading.

FIG. 20 is a series of photomicrographs showing Nile red staining in 6-day old adult wild-type, kat-1 tub-1 double mutants, and kat-1 tub-1; egl-4 triple mutants.

FIG. 21 is a series of photographs of worm culture plates showing the paths traveled by egl-4(mg410) gain-of-function and egl-4(n478) loss-of-function mutant nematodes over the course of 21 hours at 20° C. These panels illustrate the differences between dwelling and roaming phenotypes.

FIG. 22 is a series of photomicrographs showing Nile red staining in wild-type worms and in tax-2, kat-1; tax-4, and kat-1; tax-2 mutant worms.

FIG. 23 is a graph showing the quantification of Nile red fluorescence in 6-day old adult worms. The level of fluorescence of wild-type worms is normalized to 1 and the fold change in fluorescence in mutant worms is shown. The number of worms used to quantitate fluorescence is indicated in the column designated “N.”

FIGS. 24A-24D are a series of photomicrographs showing C1-Bodipy-C12 staining of 6-day-old adult wild-type and kat-2 mutant C. elegans. Giant lipid vesicles present in the kat-2 mutant are indicated with white arrows.

FIGS. 25A-25D are photomicrographs showing wild-type worms and kat-1 tub-1, and kat-2 mutant worms stained with Nile red. FIG. 25C is a photomicrograph showing a kat-2 mutant visualized with Nomarski optics. Giant lipid vesicles present in the kat-2 mutant are indicated with white arrows.

FIGS. 26A-26D are photomicrographs of wild-type L4 C. elegans. FIGS. 26A and 26C are nematodes visualized using Nomarski optics. FIGS. 26B and 26D show altered GFP-PTS1 (peroxisome target sequence) expression in kat-2 mutant nematodes. In these mutants, peroxisomes are clustered together and are in close proximity to giant lipid vesicles.

FIG. 27 is a photomicrograph of a kat-2 mutant nematode visualized using Nomarski optics and fluorescence to show the altered peroxisome distribution.

### DESCRIPTION OF THE INVENTION

The present invention features nucleic acid and amino acid sequences that function in lipid accumulation and screening methods for identifying additional genes that function in a lipid accumulation pathway.

As described in more detail below, C. elegans models that recapitulate the phenotypes of tubby mice and human Bardet-Biedl syndrome were identified using forward and reverse genetic screens. Because key mechanisms of body fat and sterol regulation are conserved between mammals and C. elegans, the powerful genetics and genomics of C. elegans can be exploited for the systematic identification of new genes as well as for the high throughput screening of candidate compounds that modulate lipid accumulation, obesity, and obesity-related diseases.

C. elegans tub-1 and bbs-1 are required for the functional and structural integrity of ciliated neurons, respectively. tub-1 and bbs-1 function in the same genetic pathway and loss-of-function mutations in either gene leads to a mild increase in lipid accumulation that can be dramatically enhanced when peroxisomal very-long-chain fatty acids β-oxidation is blocked in non-neuronal tissues by mutations in kat-1. Furthermore, occlusion of amphid ciliated neurons from the external environment in daf-6 mutant worms also causes overt lipid accumulation upon inactivation of kat-1. Our results highlight the role of ciliated neurons in the control of global lipid homeostasis and provide a framework for how environmental signals such as food availability may be directly coupled to fatty acid metabolism in peripheral tissues.

tub-1

The mammalian tub gene encodes an evolutionarily conserved protein that is highly expressed in the central nervous system, notably in the hypothalamus, a centre of feeding control. tub is also transcribed in the spiral ganglion of the inner ear and in the photoreceptor cells of the retina. Loss of function mutations in murine tub lead to late-onset obesity, retinal degeneration and hearing loss in tubby mice. The phenotypes of tubby mice resembles that of patients with Alstrom and Bardet-Biedl syndromes, although no mutation in the human tub homologue has been found that is associated with late onset obesity and neurodegeneration. Nevertheless, mutations in Tulp1, a tubby gene family member, are the cause of one form of retinitis pigmentosa (RP14), thus underscoring the importance of this gene family in human health and disease.

Despite the conservation of the tubby gene family and the well documented phenotypes of tubby mice, the cellular role and the molecular function of the TUB protein family remain to be ascertained. The TUB-like protein 1 (TULP1) has been implicated in intracellular vesicular transport of rhodopsin in the retina. It has been reported that TUB can be phosphorylated by the insulin receptor, and its subcellular localization can be modulated by G-protein coupled receptor signaling. It is unclear how these observations are related to the role of TUB in regulation of body weight.

To better understand the molecular function of TUB requires the identification of additional genes that act downstream of tub. These tub downstream genes may mediate tub function in the nervous system or act distally in peripheral tissues to affect energy balance and fat metabolism in response to neuronal tub activity. There is a single tub homologue in C. elegans, thus avoiding complications from functional redundancy when multiple gene family members are present. More importantly, C. elegans is amenable to large scale genetic and functional genomic screens which are not feasible in mice. Because genes that regulate fat storage and metabolism are highly conserved from C. elegans to human, genes that function downstream of tub-1 in C. elegans are likely to provide therapeutic targets for the treatment of human obesity.

### Conservation of Function and Expression Pattern of tub-1

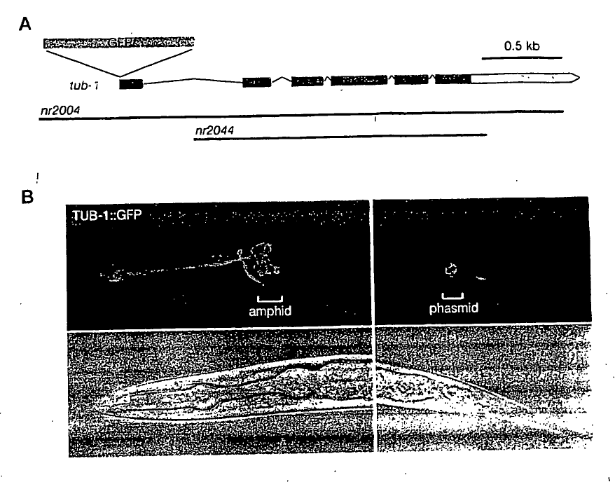

A single predicted open reading frame of the tubby gene family, originally designated F10B5.4 and renamed tub-1, was identified in the genomic sequence of C. elegans. As is the case for other members of this gene family, sequence conservation is evident only in the carboxy-terminal half of the protein. To determine the expression pattern of tub-1, a tub-1::GFP fusion gene was generated that included 1.5 kb of the tub-1 5′ regulatory region, the entire tub-1 coding sequence and introns, fused to the coding sequence of green fluorescence protein (GFP) immediately 5′ to the initiator codon of tub-1 (FIG. 1A).

TUB-1::GFP expression was highly expressed in the ciliated amphid neurons in the head and in the ciliated phasmid neurons in the tail (FIG. 1B). Lower levels of expression were observed in neurons whose locations are consistent with those of other ciliated neurons in the worm. GFP expressing neurons in the head, anterior to the amphid neurons, were tentatively identified as the inner and outer labial neurons. In addition, GFP expression was observed in neurons identified as ADE (a pair of bilaterally symmetric neurons in the deirid sensillum), AQR (an asymmetric neuron in the head), PDE (a pair of bilaterally symmetric neurons in the postdeirid sensillum), and PQR (an asymmetric neuron in the tail). The TUB-1::GFP fusion protein was localized to the neuronal cell body, axons, dendrites and ciliated endings, but was excluded from the nucleus.

tub-1 Deletion Mutants

To determine the function of tub-1, a PCR-based screening approach was used to isolate two independent tub-1 deletion mutations (FIG. 1A). The tub-1(nr2004) deletion, isolated from a strain carrying a Tc1 transposon insertion allele, removed 3.2 kb of nucleic acid sequence that spanned the entire tub-1 open reading frame. The tub-1(nr2044) deletion, isolated from a library of worms that were mutagenized with diepoxyoctane, removed 2.2 kb of nucleic acid sequence that spanned all but the first of the six exons of the tub-1 gene. Given the extent of these deletions, tub-1(nr2004) and tub-1(nr2044), which display similar mutant phenotypes, likely constitute molecular null alleles.

### Increased Lipid Levels Accumulate in tub-1 Mutant Worms

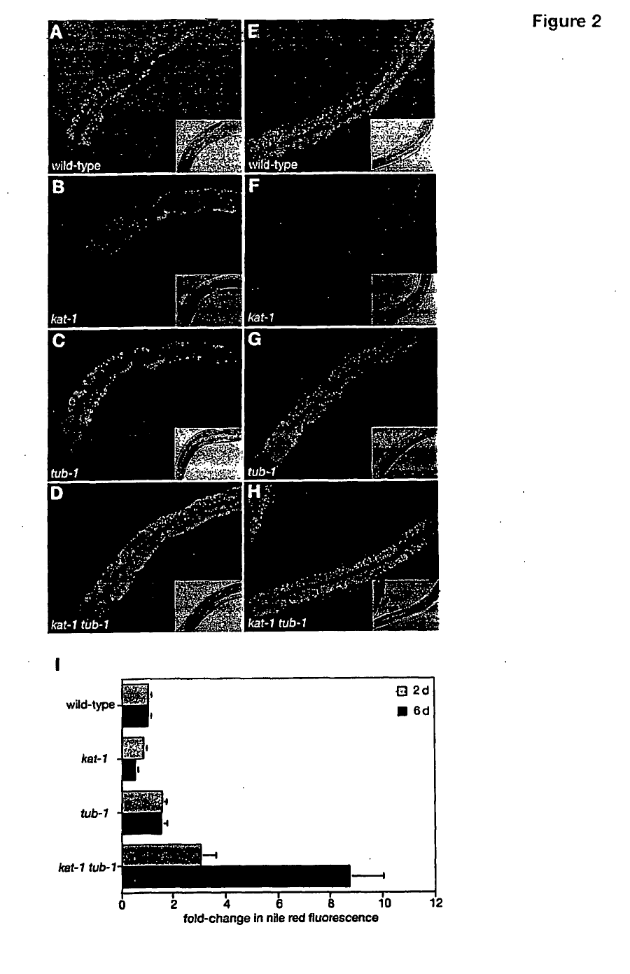

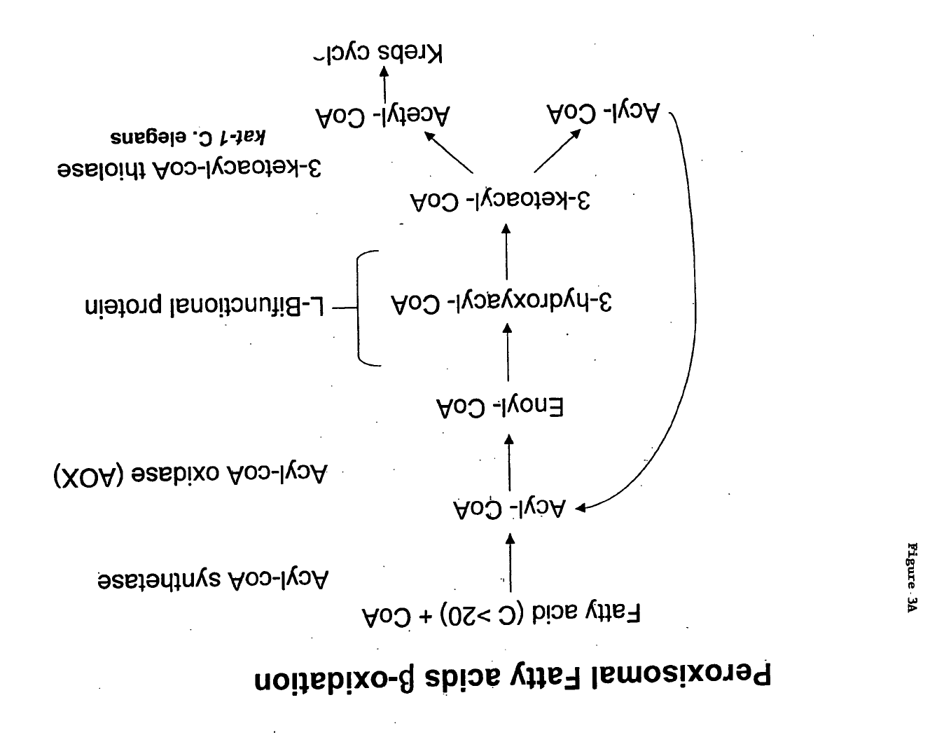

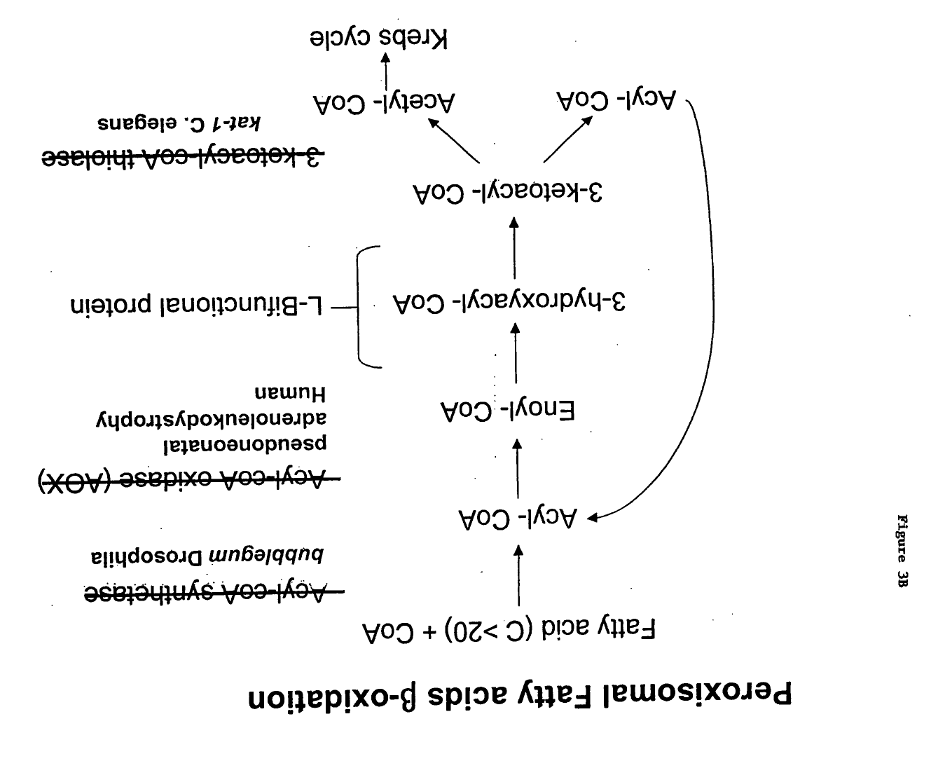

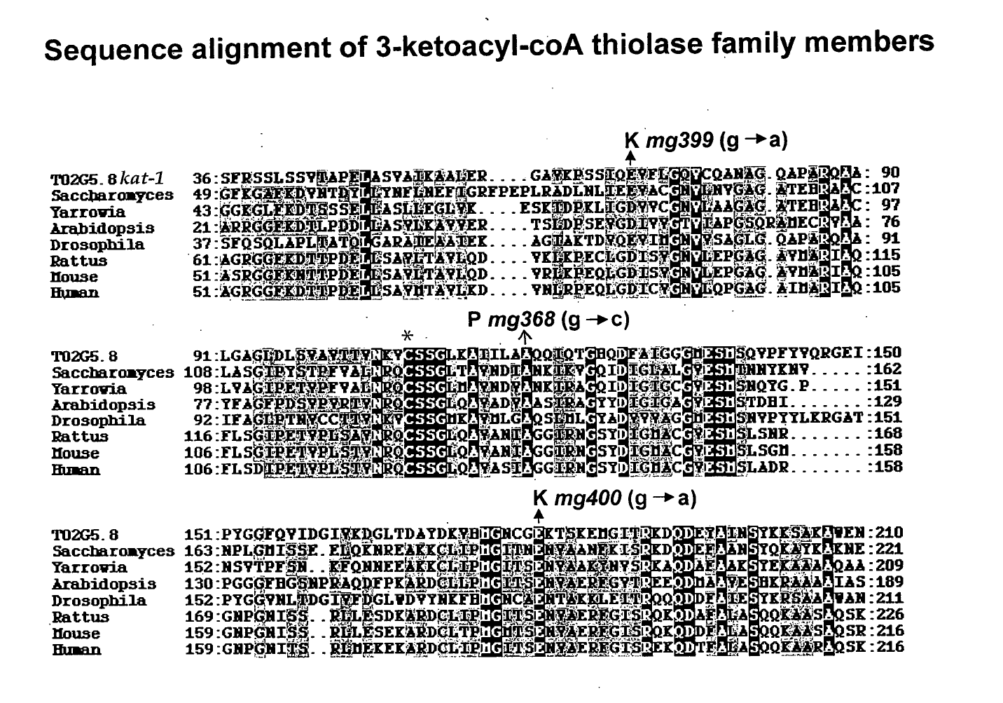

Because tubby mutant mice are obese, tub-1(nr2004) mutant worms were tested to determine whether they accumulated increased levels of lipid relative to wild-type worms. The vital dye Nile red, which causes neutral lipids to fluoresce, was used to examine lipid accumulation in the intestine, the major lipid storage organ in C. elegans (Ashrafi et al., Nature 421, 268-272, 2003) (FIGS. 2A-2H). Nile red fluorescence was increased 1.5-fold in tub-1(nr2004) worms at both reproductive and post-reproductive stages relative to wild-type worms (FIG. 2C). This increase in lipid accumulation in tub-1(nr2004) worms was confirmed with BODIPY labeled fatty acid analogue staining (C1-BODIPY-C12) (FIG. 2G), which was also increased. Quantitation of these results is shown in FIG. 2I. These results indicated that C. elegans tub-1 and mammalian tubby are conserved throughout evolution and function in the control of lipid homeostasis by ciliated neurons.

### Identification of kat-1, a tub-1 Interacting Gene

A forward genetic screen was performed to identify genes that are involved in or that interact with the tubby pathway. Five mutants were identified that confer a dramatic increase in lipid accumulation in tub-1(−) worms, as indicated by staining with the vital dye Nile red. These five mutants, mg368, mg399, mg400, mg401 and mg402, were phenotypically indistinguishable and failed to complement each other, thus defining a single gene. mg368 tub-1(−) double mutant worms displayed an approximately 6-fold increase in lipid content when compared with tub-1(−) or mg368 mutant worms, and a 9-fold increase in lipid content relative to a wild-type control. The severity of the mutant phenotype was age dependent and was most prominent in post-reproductive worms.

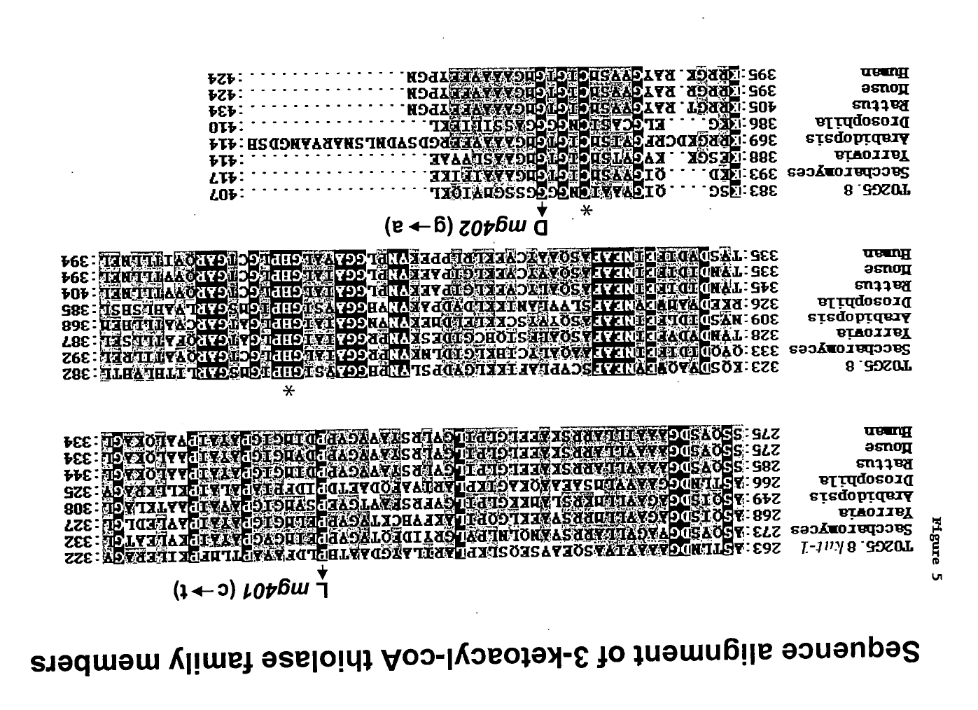

The excess accumulation of lipid did not compromise the reproductive development or lifespan of the mutant worms. The pharyngeal pumping rate and defecation cycle of well-fed worms also appeared to be normal, indicating that the increase in lipid accumulation was not due to an increase in food consumption or retention. By SNP (single nucleotide polymorphism) mapping and cosmid rescue, the gene mutated in mg368 was cloned and found to encode a peroxisomal 3-ketoacyl-coA thiolase (kat-1) (T02G5.8), an enzyme involved in β-oxidation of very long chain fatty acids (Reddy and Hashimoto, Annu Rev Nutr. 21:193-230, 2001). A peroroxisomal beta-oxidation pathway and enzymes mutated in other species is shown in FIGS. 3A and 3B. Peroxisomal 3-ketoacyl-coA thiolase is a highly conserved enzyme that is conserved from yeast to humans (FIGS. 4 and 5). The C. elegans KAT-1 is 30% identical and 48% similar to human ACAA1 (NP_{—}001598). An alignment of S. cerevisiae, Arabidopsis, rat, mouse, human, Drosophila, and worm kat-1/ACAA1 revealed the following invariant motif: 108-CSSGL, where Cys108 in worms is one of the three catalytic residues of this enzyme. Although the BLASTP score for human ACAT1 or ACAA1 against worm KAT-1 is similar, human ACAT1 does not have the motif although the catalytic cys is present.

### KAT-1 Includes a Peroxisomal Matrix Targeting Signal

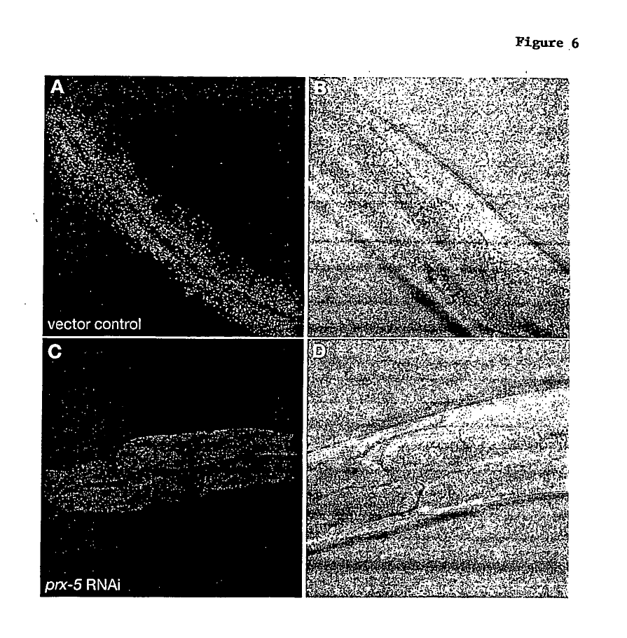

To determine whether KAT-1 includes a functional peroxisomal matrix targeting signal (PTS1), the last 7 amino acids of KAT-1 were appended to the C-terminus of GFP (FIGS. 6A-6D). When this GFP fusion protein was expressed in the C. elegans intestine, it localized to punctate cytoplasmic structures characteristic of peroxisomes. This localization was disrupted when peroxisomal import was disrupted by RNAi against the prx-5 gene, which encodes a peroxisomal matrix protein transporter (Petriv et al., Physiol Genomics 10, 79-91, 2002; Thieringer et al., J Cell Sci 116, 1797-1804, 2003). Following prx-5 RNAi, diffuse GFP fluorescence was observed (FIGS. 6A and 6C).

### Conserved Amino Acids are Altered in kat-1 Mutants

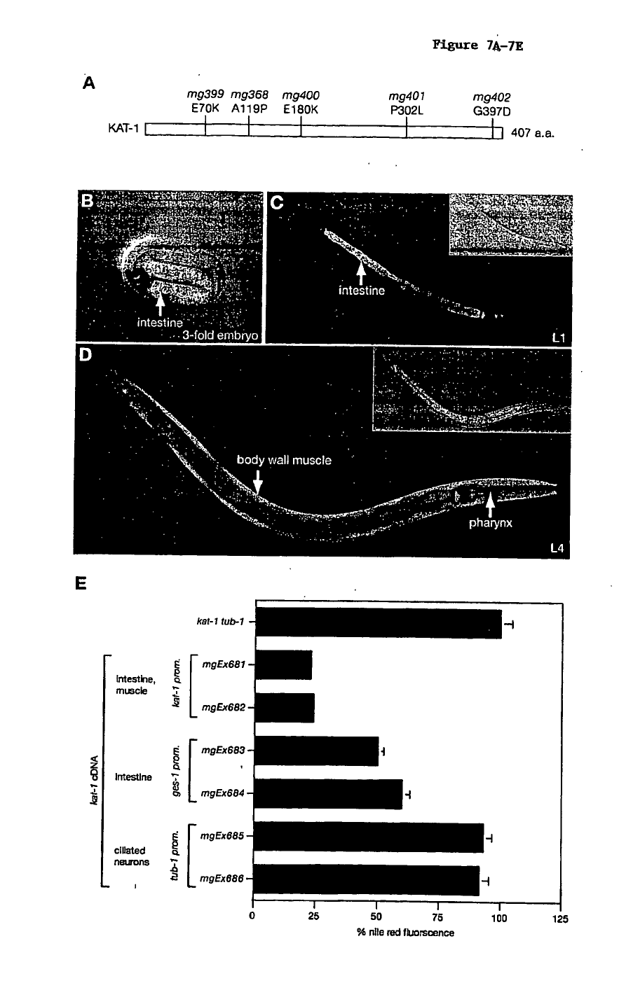

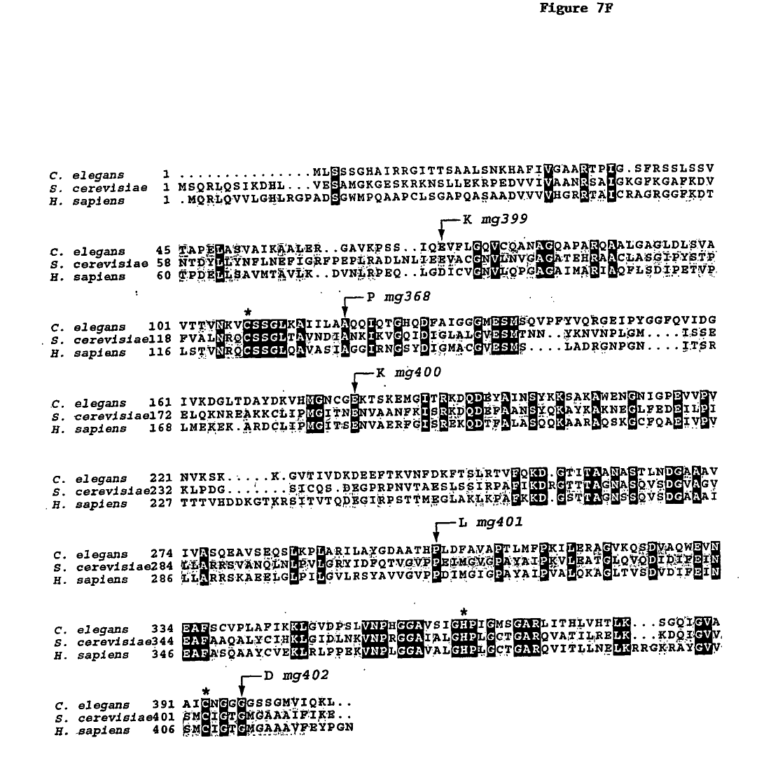

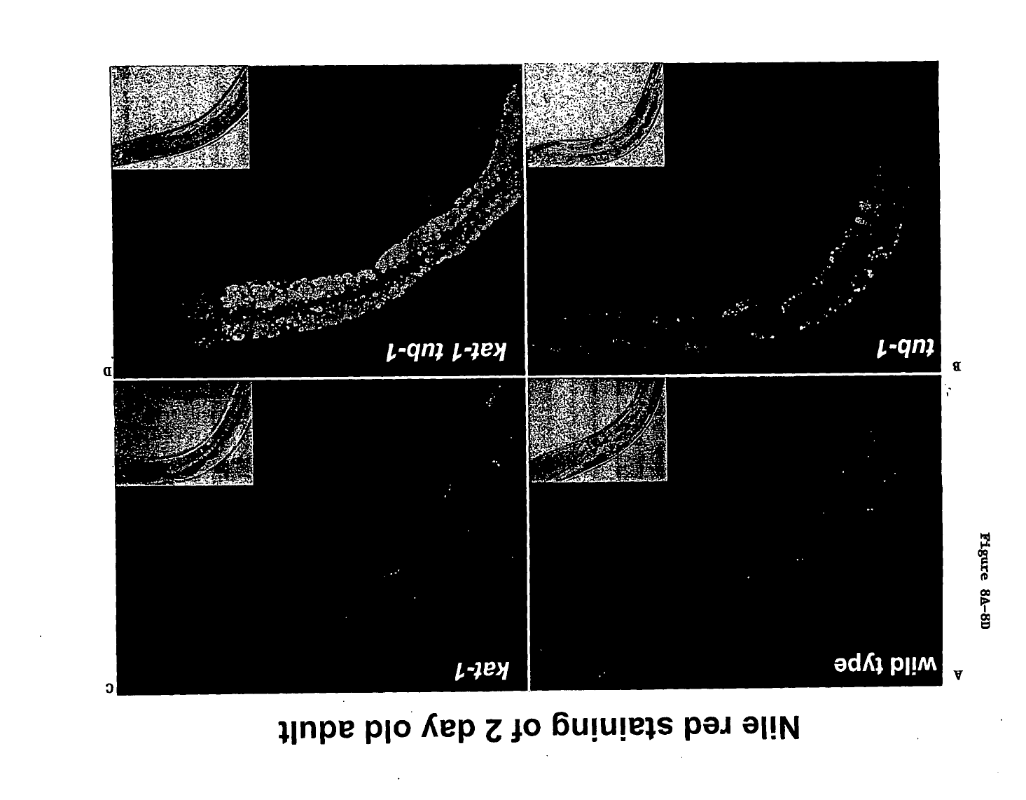

Sequencing of the kat-1 coding region in the five mutant alleles identified missense mutations that resulted in the substitution of highly conserved amino acid residues (FIGS. 7A and FIG. 7F). For example, in the mg368 mutation alanine 119 was converted to proline. This mutation, which resulted in a severe loss-of-function phenotype, likely disrupts an α-helix that is critical for the architecture of the catalytic site and dimerisation property of the enzyme, based on the crystal structure of its yeast homologue, Potip (Mathieu et al., J Mol Biol 273:714-728, 1997). The mutations in other kat-1 alleles are predicted to disrupt the KAT-1 catalytic site and/or amino acids required of KAT-1 dimerisation.

kat-1 Expression Pattern

To determine the expression pattern of kat-1, a transcriptional GFP fusion gene, kat-1p::GFP, was constructed. Expression of kat-1p::GFP was detected in the intestine from late embryogenesis (FIGS. 7B and 7C), and in the body wall muscle and pharynx from larval stage 2 (L2) (FIG. 7D). Weak and inconsistent kat-1p::GFP expression was also observed in at least two pairs of unidentified head neurons.

### Characterization of kat-1(mg368) Phenotype

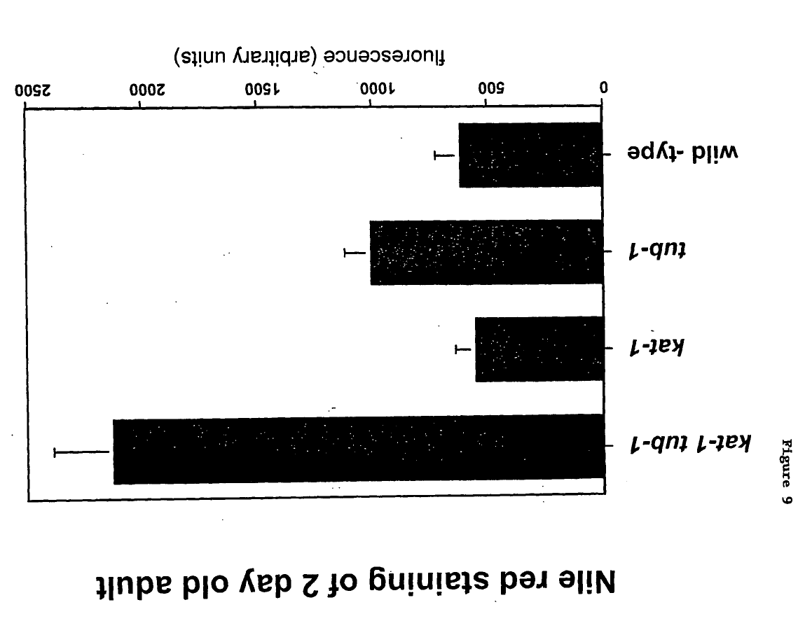

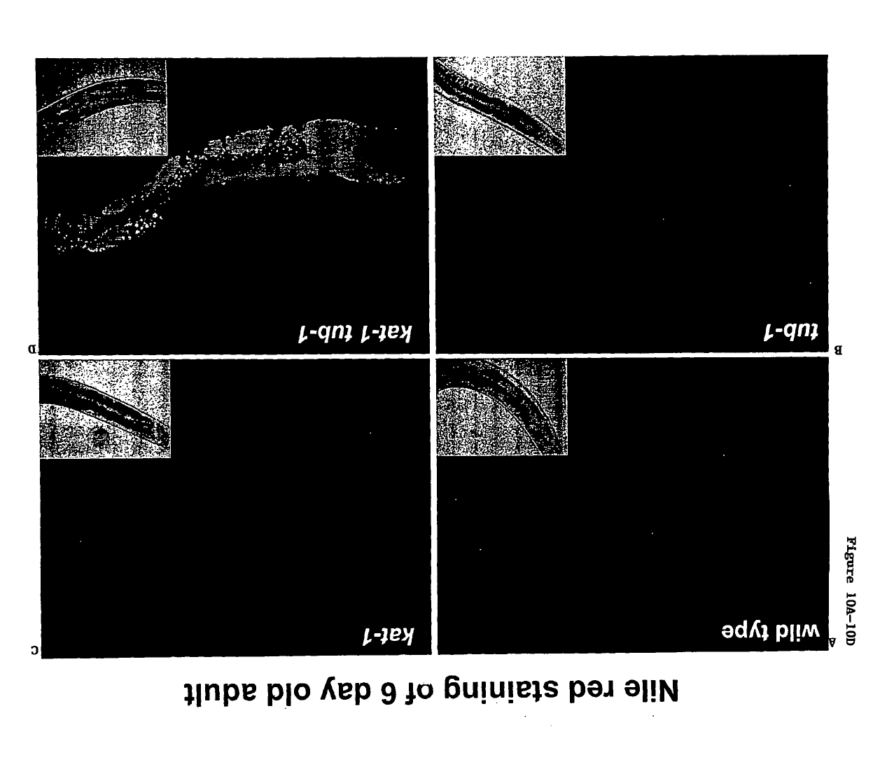

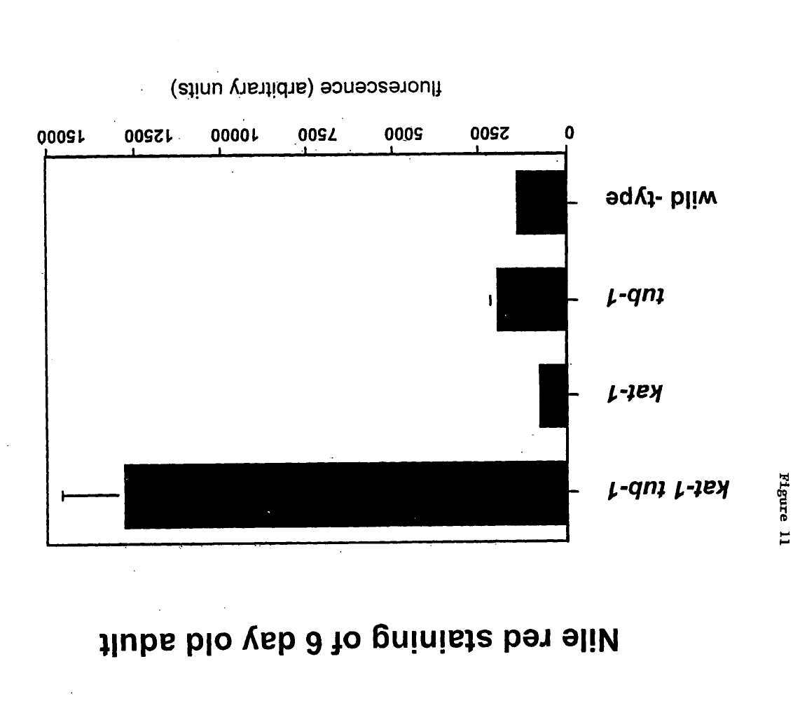

kat-1(mg368) mutant worms showed a transient elevation of lipid content as young adults as detected by Nile red staining. This was followed by rapid depletion of lipid content to a level below that of wild-type control in 2 day old adults. FIG. 8A-8D show Nile red staining of wild-type, kat-1, tub-1 and kat-1; tub-1 mutant nematodes in 2 day old adults and the quantitation of this staining is shown in FIG. 9. FIG. 10 shows Nile red staining in 6 day old adults, and the quantitation of this staining is shown in FIG. 11. The accumulation of reaction intermediates in kat-1(+) dependent β-oxidation pathway triggers feedback upregulation of parallel β-oxidation pathways in peroxisomes and mitochondria that lead to a depletion of fat storage.

### Tissue Specific Expression of kat-1

Because there was no significant overlap in the expression patterns of kat-1 and tub-1, the expression pattern of the two genes did not suggest a mechanism by which they interacted to control fat accumulation. To determine the site of action of kat-1, kat-1(+) activity was expressed under the control of tissue specific promoters, allowing KAT-1 activity to be restricted to specific tissues (FIG. 7E). Expression of kat-1 under the control of its own promoter reduced the Nile red staining of kat-1 tub-1 worms by 75%. When kat-1 was expressed in the gut under the control of the ges-1 promoter, a 50% reduction in Nile red staining was observed. When kat-1 was expressed under the control of the tub-1 promoter no reduction in Nile red staining of kat-1 tub-1 worms was observed. These results indicated that loss of kat-1 activity in the gut was partially responsible for the fat accumulation phenotype observed in the kat-1 tub-1 worms. In addition, the distinct sites of action of kat-1 and tub-1 suggested that global lipid homeostasis is likely to be controlled by tub-1 expressing neurons that produce neuroendocrine signals that are received by kat-1 expressing peripheral tissues.

kat-1 and tub-1 Mutations Do Not Cause Gross Abnormality in Peroxisomes and Mitochondria

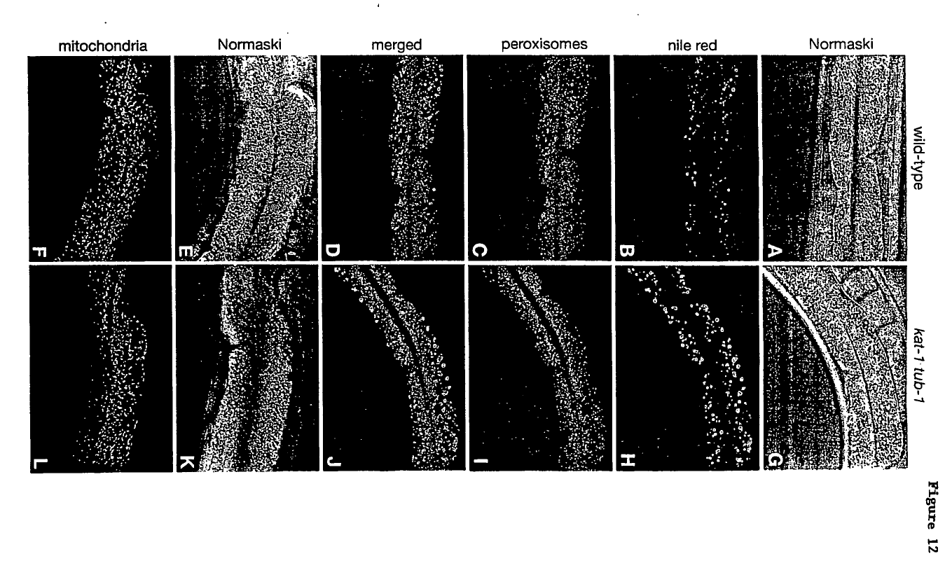

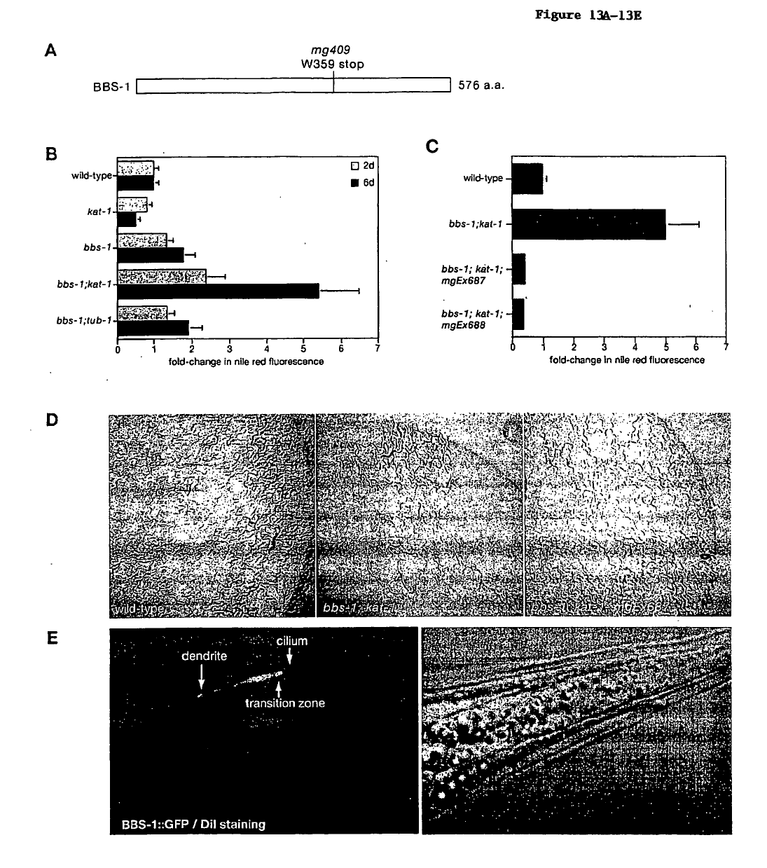

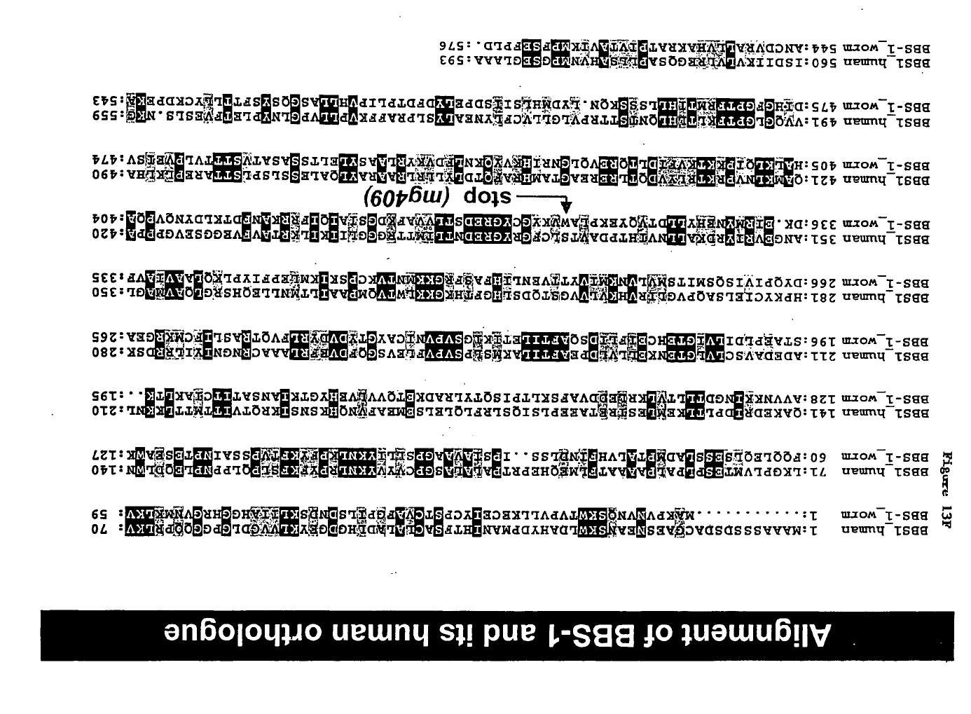

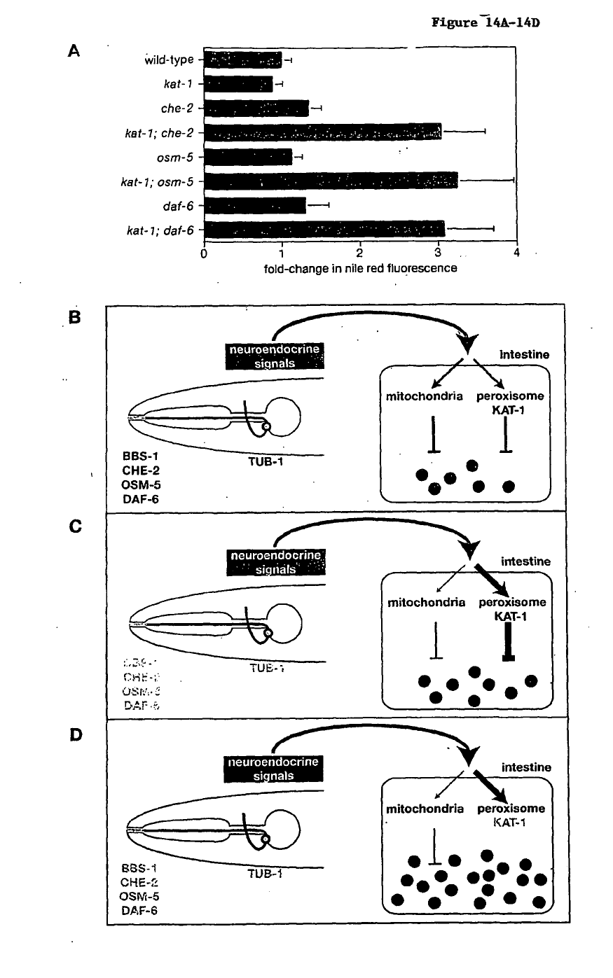

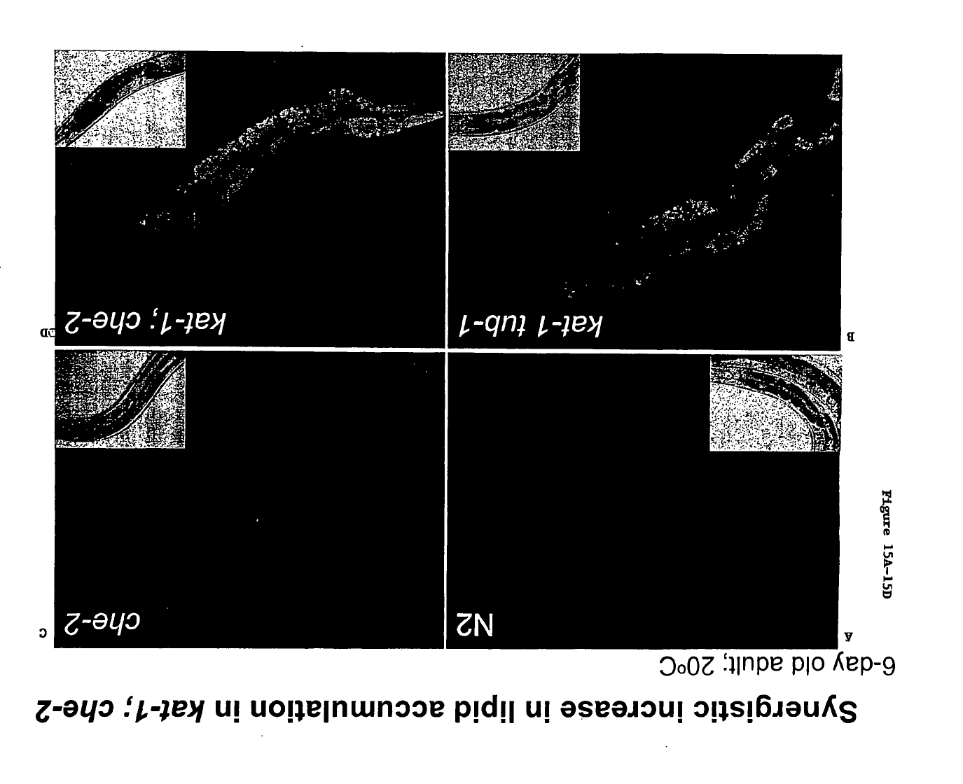

The KAT-1 enzyme acts at the last step in the β-oxidation of very-long-chain fatty acids in peroxisomes (Reddy and Hashimoto, Annu Rev Nutr. 21:193-230, 2001). Inactivation of KAT-1 is likely to result in the accumulation of fatty-acyl-coA and other β-oxidation intermediates in the peroxisomes. To test whether such an accumulation compromises peroxisomal function and leads to alterations in peroxisomal morphology or number, a transgene that directed intestine-specific expression of a peroxisomal matrix targeted GFP was expressed in wild-type and kat-1 tub-1 worms (FIGS. 12A-12L). No significant difference in peroxisomal morphology or number was detected by confocal microscopy (FIGS. 12C and 12I). When kat-1 tub-1 worms were stained with Nile red, minimal overlap was observed between peroxisomal GFP and Nile red fluorescence signals (FIGS. 12D and 12J), indicating that no neutral lipids accumulated in the peroxisomes of kat-1 tub-1 mutant worms.

To test whether a block in peroxisomal β-oxidation led to a compensatory upregulation of mitochondrial β-oxidation that resulted in an alteration in mitochondrial morphology, the morphology of intestinal mitochondria was analyzed in wild-type and kat-1 tub-1 mutant worms (FIGS. 12F and 12L). No alterations in mitochondrial morphology were observed, suggesting that kat-1 and tub-1 mutations do not cause gross disruptions of peroxisomal and mitochondrial function. This is consistent with the lack of pleiotropic phenotypes in kat-1 tub-1 mutant worms.

### Cloning of C. elegans Orthologue of Bardet-Biedl Syndrome Gene BBS1

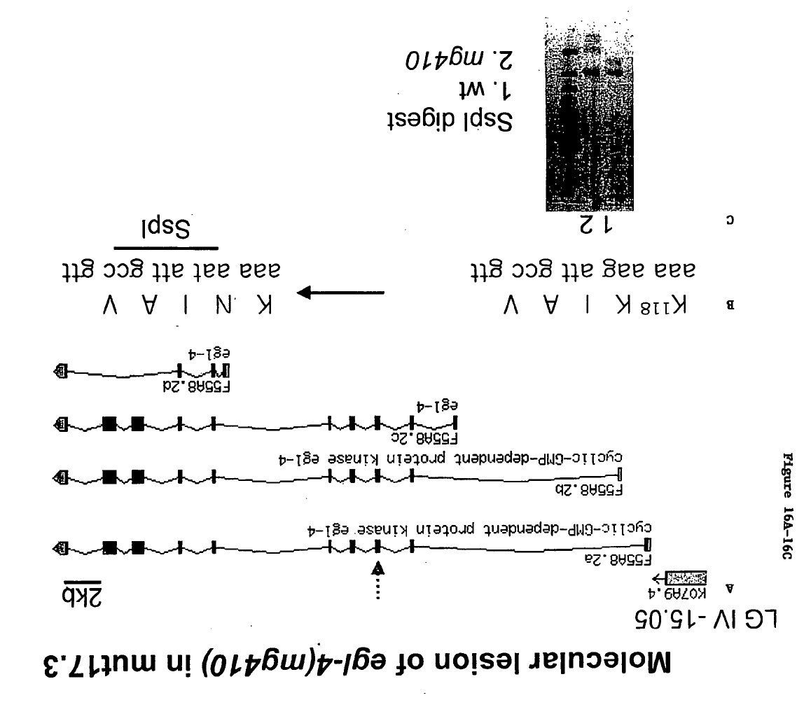

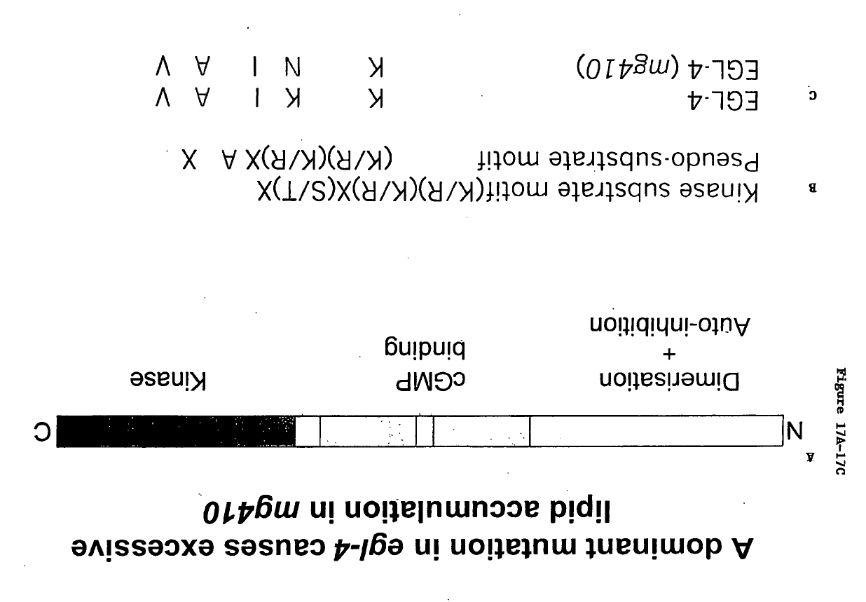

The specificity of the genetic interaction between tub-1 and kat-1 was demonstrated by the number of independent alleles of kat-1 that were isolated from the tub-1(nr2004) genetic modifier screen. No other isolates from this screen yielded the same lipid accumulation phenotype as kat-1 tub-1 mutant worms. The specificity of the genetic interaction between tub-1 and kat-1 suggested that additional components of the tubby pathway might be identified in a kat-1(mg368) modifier screen. To this end, 79,200 haploid genomes were screened after EMS mutagenesis of kat-1(mg368) worms, to identify mutants that showed a synergistic increase in lipid accumulation in a kat-1(mg368) dependent manner. 41 independent mutants were recovered from this screen, including mg409 (FIG. 13A). Worms that have mutations in both mg409 and kat-1 (mg409; kat-1(mg368) mutant worms) have a 5-fold increase in lipid accumulation relative to wild-type post-reproductive adult worms (FIG. 13B). The mg409 and kat-1(mg368) mutations were highly synergistic since mg409 worms showed a mere 1.5-fold increase in lipid accumulation when compared to wild-type worms. In addition, mg409 mutant worms are dye filling defective (Dyf), indicating a failure in ciliated neuron differentiation. In addition, mg409 worms have a small body size (Sma) and display excessive dwelling behavior, two phenotypes that are likely the result of sensory deficits (Fujiwara et al., Neuron 36:1091-1102, 2002).

### Cloning of mg409

The gene mutated in mg409 was identified by SNP mapping as an ortholog of human BBS1. Human bbs-1 is a gene that is often mutated in Bardet-Biedl syndrome patients. Bardet-Biedl syndrome is a complex genetic disease that includes obesity as a prominent clinical features (Katsanis et al., Hum Mol Genet 13 Spec No 1, R65-71, 2001). In C. elegans, the bbs-1(mg409) (Y105E8A.5) mutation converts the codon for tryptophan 359 to a stop codon (FIGS. 13A and 13F). Introduction of a transgene spanning the genomic region of bbs-1 rescued the dye filling defective, dwelling, and excessive lipid accumulation phenotypes of bbs-1(mg409); kat-1(mg368) worms (FIGS. 13C and 13D). Similar to tub-1, bbs-1 was expressed in ciliated neurons in C. elegans (Ansley et al., Nature 425:628-633, 2003). While it is possible that tub-1 and bbs-1 function in two parallel pathways that genetically interact with kat-1, it seems likely that bbs-1 and tub-1 function in the same genetic pathway, because bbs-1(mg409); tub-1(nr2004) double mutant worms do not accumulate more neutral lipid than bbs-1(mg409) and tub-1(nr2004) single mutant worms (FIG. 13B).

Many BBS gene family members localize to the basal body of primary cilium (Ansley et al., Nature 425:628-633, 2003; Kim et al., Nat Genet 36:462-470, 2004). To determine whether BBS-1 displayed a similar subcellular localization, a transgene was generated to drive the expression of a GFP-tagged BBS-1 fusion protein in ciliated neurons under the control of the che-2 gene promoter (Fujiwara et al., Development 126:4839-4848, 1999). This transgene rescued the lipid accumulation phenotype of bbs-1; kat-1 mutant worms (FIG. 13C), suggesting that the BBS-1::GFP fusion protein was fully functional. Similar to other BBS proteins, the BBS-1::GFP fusion protein was localized to the transition zone of C. elegans ciliated neurons (FIG. 13E), a structure analogous to the basal body in other organisms. GFP fluorescence in punctate structures of the neuronal cell body was also present. These results indicated that multiple distinct BBS proteins may localize to the same subcellular structure.

### Lipid Accumulation in Worms with Abnormal Sensory Cilia

The structural defect present in ciliated neurons in bbs-1 mutant worms may cause excess lipid accumulation in kat-1(−) worms. Alternatively, bbs-1 may act in concert with tub-1 to control lipid accumulation, independent of its role in ciliated neuron differentiation. Besides bbs-1, a number of genes are known to be required for the differentiation and maintenance of ciliated neurons. To determine whether structural defects of ciliated neurons per se can lead to excessive lipid accumulation in kat-1(mg368) worms, kat-1(mg368); che-2(e1033) and kat-1(mg368); osm-5(p813) worms mutant worms were stained with Nile red.

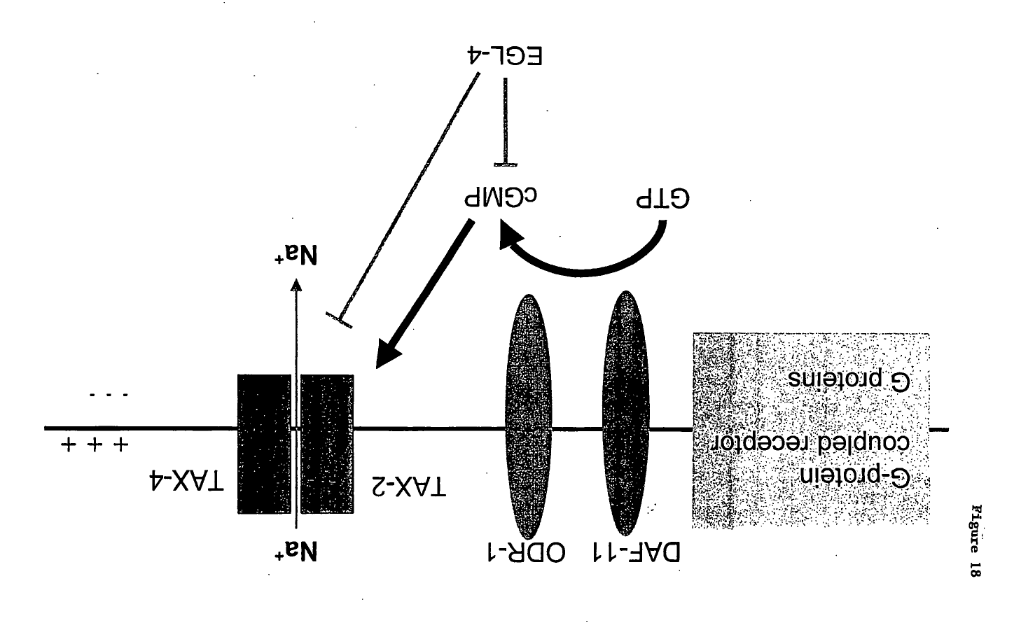

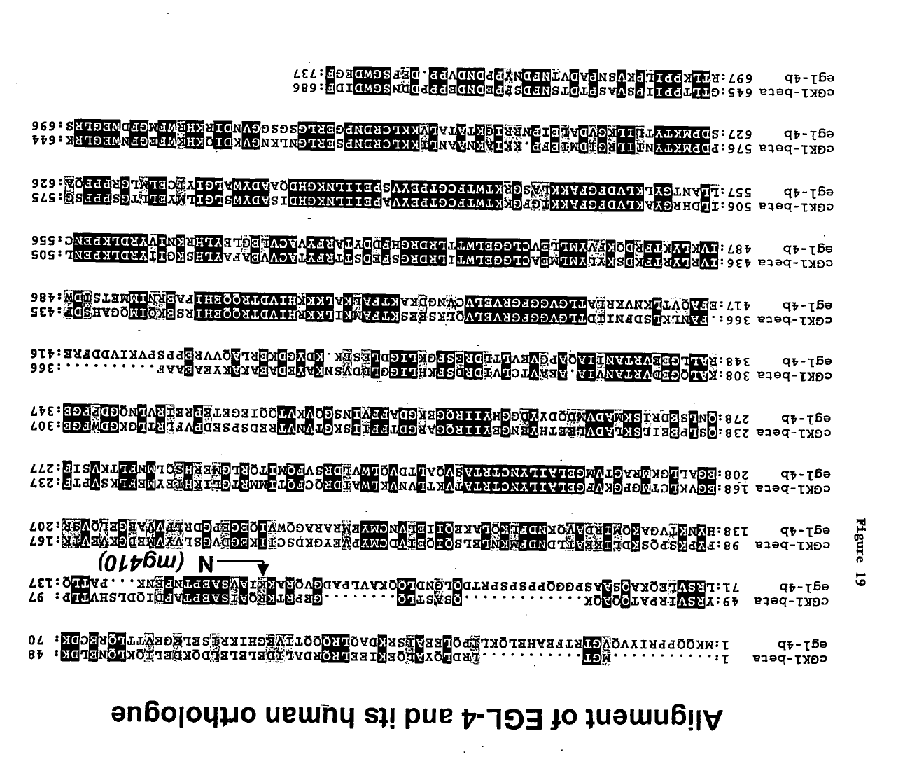

While che-2(e1033) and osm-5(p813) single mutant worms do not accumulate excessive lipid, kat-1(mg368); che-2(e1033) and kat-1(mg368); osm-5(p813) worms displayed phenotypes similar to bbs-1(mg409); kat-1(mg368) mutants (FIG. 14A). As 2-day old adult worms, these mutants all accumulated up to 3-fold more lipid than wild-type worms. Nile red staining is shown in FIGS. 15A-15D for N2, che-2, kat-1, tub-1, and kat-1; che-2 mutant worms. These results indicated that control of lipid accumulation depends on the structural integrity of ciliated sensory neurons in C. elegans, as exemplified by bbs-1, che-2 and osm-5 mutant worms.

Lipid accumulation was examined in daf-6(e1377) mutant worms that have a defect in the non-neuronal amphidial sheath cell. This defect leads to the obstruction of the amphid channel (Albert et al., J Comp Neurol 198:435-451, 1981; Herman, Genetics 108:165-180, 1984). Even though there is no defect in ciliated amphid neuron formation, the neurons are not exposed to the external environment in daf-6(e1377) mutant worms. While daf-6(e1377) mutant worms appeared normal, excessive lipid accumulation was observed in kat-1(mg368); daf-6(e1377) mutants (FIG. 14A). These results indicated that amphid sensory neurons play an important role in lipid homeostasis, possibly by sensing food availability. Models for how amphid sensory neurons may function in the control of global lipid homeostasis are provided at FIGS. 14B, 14C, and 14D.

As described above, C. elegans models of tubby and Bardet-Biedl syndrome were identified through forward and reverse genetic screens. C. elegans tub-1 and bbs-1 are required for the functional and structural integrity of ciliated neurons, respectively. tub-1 and bbs-1 function in the same genetic pathway and loss of function mutations in either gene resulted in a mild increase in lipid accumulation. This lipid accumulation phenotype was dramatically enhanced when peroxisomal very-long-chairs fatty acids β-oxidation was blocked in non-neuronal tissues by mutations in kat-1. Furthermore, occlusion of amphid ciliated neurons from the external environment in daf-6 mutant worms also caused lipid accumulation when kat-1 was inactivated. These results highlight the importance of ciliated neurons in the control of global lipid homeostasis and suggest that the sensation of environmental signals, such as food availability, is coupled with fatty acid metabolism in peripheral tissues.

We speculate that ciliated neurons emit neuroendocrine signals that ultimately control fatty acid β-oxidation in peroxisomes and mitochondria of peripheral tissues. Under normal conditions, straight chain fatty acids may enter the peroxisomes to undergo limited rounds of β-oxidation. Fatty acids of chain length C<8 are then imported to the mitochondria for complete oxidation Reddy and Hashimoto, Ann Rev Nutr. 21:193-230, 2001). Although the peroxisomes play a minor role in dietary long-chain (C14-C20) fatty acids oxidation at the basal state, their number and β-oxidation activity can be strongly induced by high-fat diet or increase in free fatty acids level (Reddy and Hashimoto, Ann Rev Nutr. 21:193-230, 2001). It is conceivable that ciliated neuron dysfunction in tub-1 or bbs-1 mutant animals may lead to reduced mitochondrial β-oxidation (FIG. 14C). This is partially counteracted by compensatory increase in peroxisomal β-oxidation that strictly demands kat-1(+) activity. Mild elevation of lipid accumulation in tub-1 or bbs-1 mutant animals may arise from incomplete oxidation of fatty acids. The absence of such homeostatic response in kat-1(−) genetic background, however, results in dramatic increase in fat deposition (FIG. 14D). Mammalian peroxisomes are sites for processing multiple ligands for nuclear receptors, including PPARs, which are intimately involved in fat metabolism. It is conceivable that production of such lipophilic ligands may be compromised in kat-1(−) animals and hence reinforce the excessive lipid accumulation phenotype in kat-1 tub-1 mutant animals.

It has been reported that tubby nuclear localization can be induced by activated G-protein coupled receptor signaling that dissociates tubby from PIP₃ at the plasma membrane of mammalian neuronal culture cells (Santagata et al., Science 292: 2041-2050, 2001). Constitutive tubby nuclear localization resulted from two point mutations that disrupt the PIP₃ binding of tubby (Santagata et al., Science 292: 2041-2050, 2001). We did not observe nuclear localization when analogous mutations were introduced into a GFP-tagged TUB-1 fusion protein that was expressed in ciliated neurons under the control of tub-1 promoter. This may reflect different modes of regulation for tubby family members in C. elegans and mammals. Nevertheless, multiple lines of evidence now support a role for tubby in intracellular vesicular transport. For example, degeneration of photoreceptor cells in tub^{-/-} and tulp1^{-/-} mice has been attributed to a failure to transport rod and cone opsins to the outer segment (Hagstrom et al., Invest Ophthalmol Vis Sci 40:2795-2802, 1999; Hagstrom et al., Invest Ophthalmol Vis Sci 42:1955-1962, 2001). Abnormal accumulation of cholinergic and GABAergic synaptic vesicles was also observed in the arcuate nucleus of obese tubby mice (Backberg and Meister, Synapse 52: 245-257, 2004). In addition, the modifier of tubby hearing (moth1) phenotype gene encodes microtubule-associated protein 1A, which has been implicated in intracellular trafficking of synaptic proteins (Brenman et al., J Neurosci 18:8805-8813, 1998; Ikeda et al., Nat Genet 30:401-405, 2002b).

Disruption of peroxisomal β-oxidation and accumulation of very-long-chain fatty acids (VLCFA) has a devastating effect in humans (Wanders et al., Biochem Soc Trans 29:250-267, 2001). The human ALDP gene encodes a putative peroxisomal VLCFA transporter, while the human peroxisomal AOX gene encodes the acyl-coA oxidase, which is required for the first step of VLCFA β-oxidation. Mutations in human ALDP and AOX genes cause X-linked adrenoleukodystrophy (OMIM 300100) and pseudoneonatal adrenoleukodystrophy (OMIM 264470), respectively, neurological disorder that result from the demyelination of neurons in the central and/or peripheral nervous systems. In C. elegans, kat-1 mutant worms do not display gross neurological defect, such as uncoordinated movement, and have a normal adult lifespan. This is not unexpected, given that VLCFA is unlikely to accumulate at levels that are sufficient to disrupt peroxisomal or other cellular functions in kat-1(−) worms even when their oxidation is blocked, because E. coli, C. elegans only food source in the laboratory, contain only trace amount of VLCFA. It should also be noted that C. elegans neurons are not myelinated.

Although extreme obesity in humans can be caused by monogenic disorders, such as mutations in the leptin gene (O'Rahilly et al., Endocrinology 144:3757-3764, 2003), the majority of obesity syndromes likely result from multiple genetic variations. kat-1 loss of function mutations synergized with other mutations that compromise the structure and function of ciliated neurons in C. elegans resulting in excessive lipid accumulation phenotypes. These results suggest that genes encoding fatty acids β-oxidation pathway components and genes required for cilia biogenesis and function should be examined in targeted genome linkage scans of oligogenic obesity disorders. Indeed, the recent identification of over 200 genes that are conserved in ciliated organisms present many new candidates for genetic association studies (Avidor-Reiss et al., Cell 117, 527-539, 2004; Li et al., Cell 117:541-5522004). It is worth noting that Bardet-Biedl syndrome (BBS) is now regarded as an oligogenic disorder since there is increasing evidence that genetic modifiers can cause the severity and penetrance of the phenotype to vary widely among BBS patients (Katsanis, Hum Mol Genet 10:2293-2299, 2004; Mykytyn et al., Trends Mol Med 10:106-109, 2004). For example, individuals that have three mutations at two BBS loci display a more severe phenotype than siblings that have two mutations at a single BBS locus (Badano et al., Human Molecular Genetics 12:1651-16592003). Our observation that a kat-1 mutation in combination with a bbs-1 mutation leads to a synergistic increase in lipid accumulation strongly suggests that BBS modifiers are not limited to members of the BBS gene family.

In summary, our analysis of tub-1, bbs-1 and other mutant worms having defects ciliated neurons or in ciliated neuron support cells indicated that control of lipid homeostasis in C. elegans depends on the structural and functional integrity of ciliated neurons and their exposure to the external environment. This requirement is likely to be linked to their role in sensing nutrient availability. In mammals, the hypothalamus, which is populated with neurons that possess non-motile primary cilia, regulates lipid accumulation (Handel et al., Neuroscience 89:909-926, 1999). These primary cilia may detect peptide hormones or small lipophilic molecules that reflect the metabolic state of the worm. Primary cilia dysfunction in tubby mice and Bardet-Biedl syndrome hypothalamic neurons may result in defects in the ability of such neurons to gauge the organism's metabolic state, resulting in excess energy intake and obesity. Furthermore, variation in the hormonal signaling axis from fat storage depots to ciliated neurons, and from ciliated neurons to fat depots to control mitochrondrial versus peroxisomal β-oxidation may underlie other more common forms of obesity. These variations may not share the pleiotropic features of Bardet-Biedl syndrome if other functions of ciliated neurons were intact.

egl-4 Identification

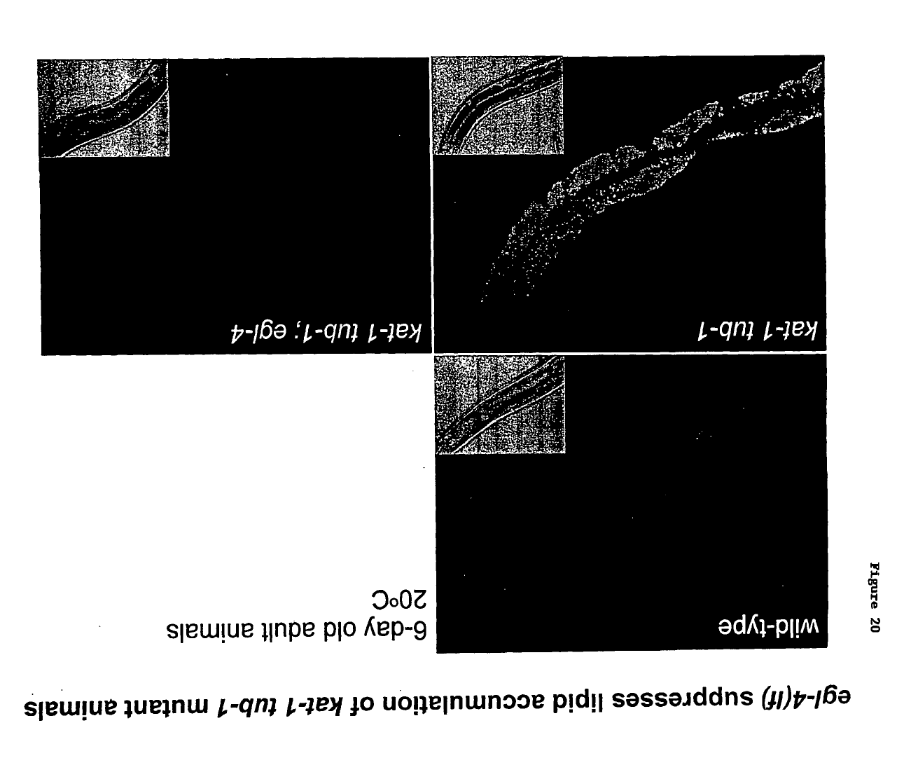

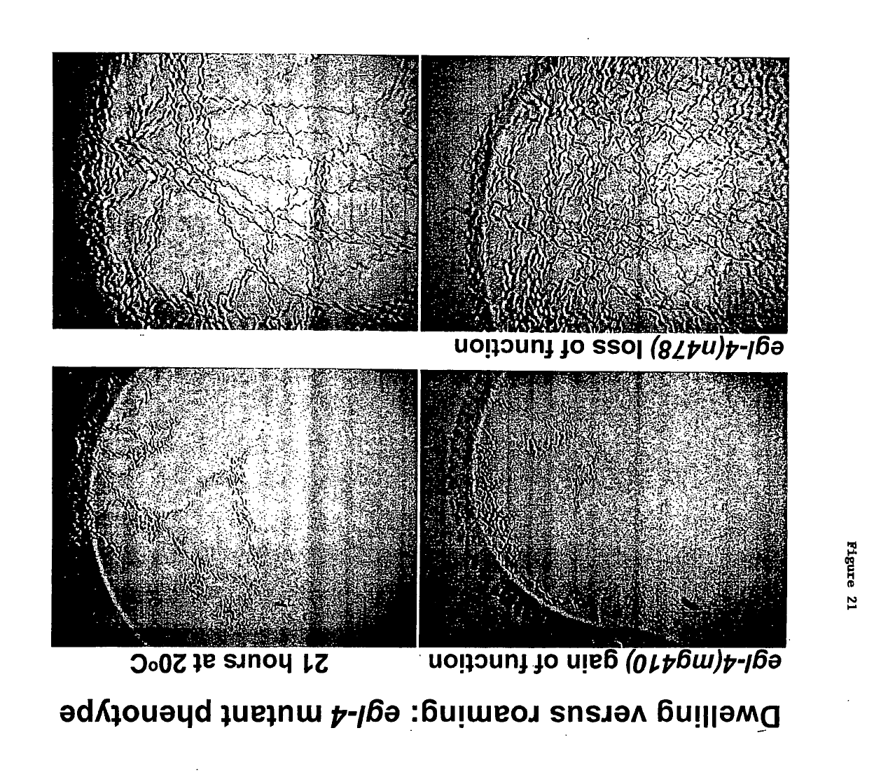

mg410 was also identified in the screen described above for mutants that show a kat-1(mg368) dependent synergistic increase in lipid accumulation (FIGS. 16A-16C). mg410 was mapped to LGIV-15.05 (16A) by SNP mapping, using methods known in the art and described herein. Because kat-1(mg368)/kat-1(mg368); mg410/+ has the same lipid accumulation phenotype as kat-1(mg368)/kat-1(mg368); mg410/mg410), mg410 was identified as a dominant mutation in egl-4, which encodes a cGMP dependent protein kinase (FIG. 16A). FIG. 17A provides a schematic diagram of EGL-4 functional domains. EGL-4 is expressed in IL1, chemosensory neurons, and in the hypodermis. Sequencing identified a mutation in mg410 that substitutes a critical Lys for Asn in the pseudo-substrate motif (FIGS. 17B and 17C). This mutation relieves the auto-inhibition of EGL-4 and leads to constitutive activation of the kinase. A schematic diagram illustrating the cGMP signaling pathway is provided at FIG. 18. An alignment of EGL-4 with its human orthologue (Genbank Accession No. P14619) is shown in FIG. 19.

### Characterization of egl-4(mg410)

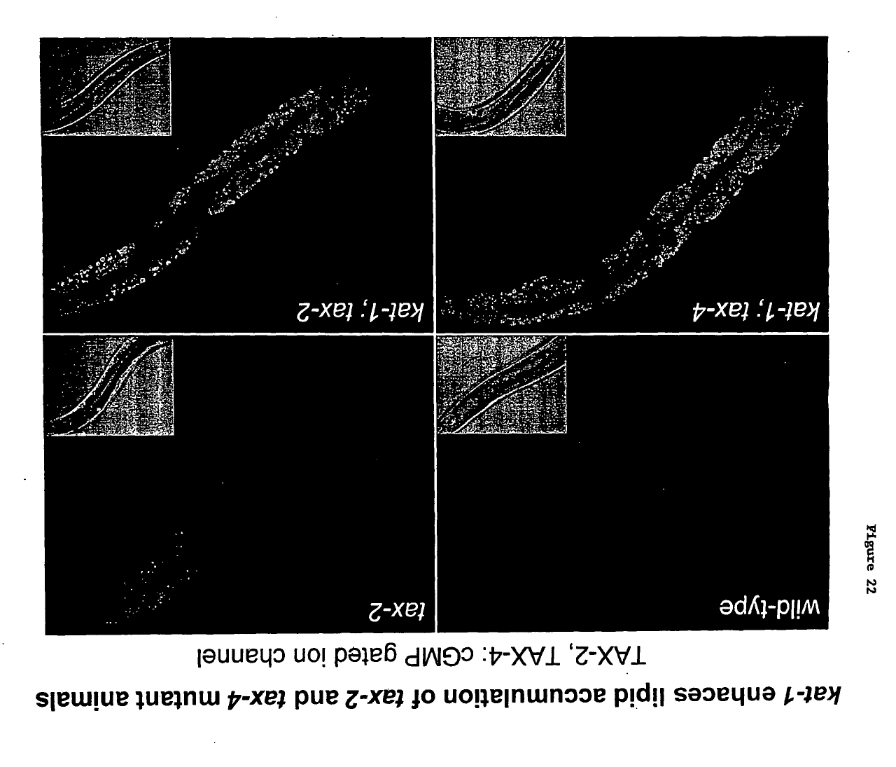

egl-4 loss-of-function is a suppressor of the che-2(e1033) small body size phenotype and of the daf-11(m47) guanyl cyclase Daf-c phenotype. Interestingly, an egl-4 loss of function mutation suppressed lipid accumulation in kat-1(mg368) tub-1(nr2004) double mutants (FIG. 20). While egl-4 loss of function mutants are characterized by chemosensory defects, a large body size, and excessive roaming, the egl-4 (mg410) gain of function allele is characterized by excessive dwelling and a small body size. FIG. 21 illustrates the dwelling versus roaming defects of egl-4 gain-of-function and loss-of-function mutant worms, respectively. Interestingly, while egl-4 (mg410) kat-1(mg368) double mutants display increased lipid accumulation,

tax-2 and tax-4 Mutations Synergize with Kat-1

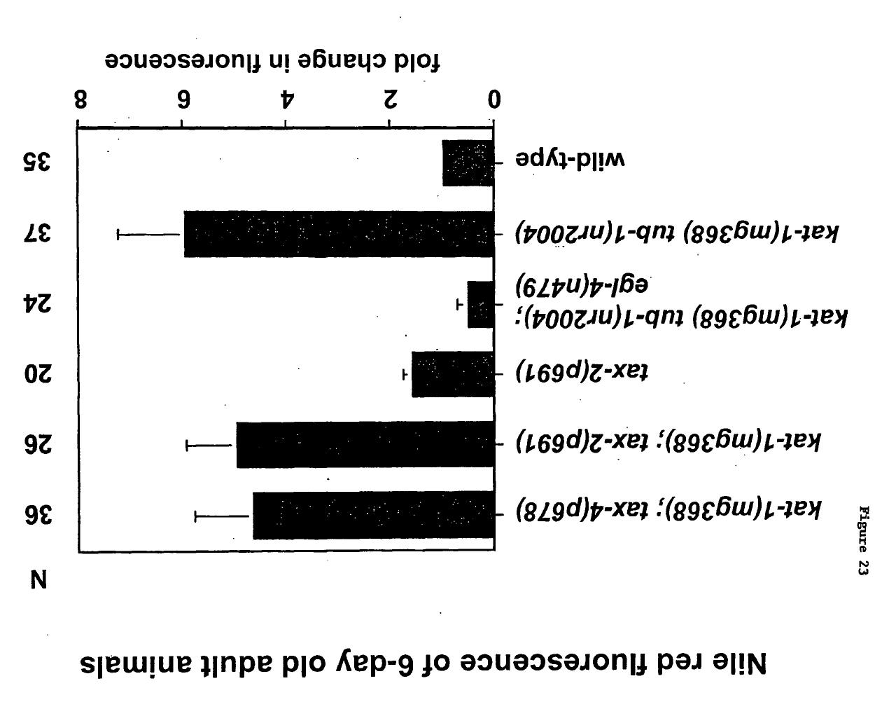

Similar to the egl-4 gain-of-function mutation, tax-2 and tax-4 loss-of-function mutations synergize with kat-1(mg368) (FIG. 22). Quantitation of Nile red fluorescence in 6-day old worms having a variety of lipid accumulation mutations is shown in (FIG. 23). tax-4 encodes a cGMP gated ion channel alpha subunit. tax-2 encodes a cGMP gated cation channel beta subunit. EGL-4 negatively regulates TAX-2 and TAX-4. These results suggest that lipid accumulation is regulated by ciliated sensory neurons and the cGMP pathway.

### Genetic Screen to Identify Genes that Act Downstream of C. elegans Tub-1

A forward genetic screen was performed by mutagenising kat-1(mg368) worms with EMS (ethylmethanesulfonate), and identifying F2 worms that phenocopied the enhanced lipid accumulation phenotype of kat-1 tub-1 mutant worms. The F2 worms were allowed to develop at 15° C. until the second larval stage and were then shifted to 25° C. to allow the identification of temperature sensitive lethal mutations. 2-day-old adult worms were screened to identify mutations that in the presence of kat-1(mg368) confer enhanced Nile red staining. At least 41 independent mutants were identified in this screen. To determine whether these mutants are dominant or recessive, the mutants are outcrossed to wild-type nematodes.

All 41 mutant lines were outcrossed. All of these mutant alleles depended on kat-1(mg368) mutation for their enhanced lipid accumulation phenotype. 39 of the mutant alleles have recessive mutations, and two of them carries a dominant mutation. Three of the recessive mutations were closely linked to kat-1(mg368) and are candidates for new loss of function mutations in the tub-1 locus.

To determine whether the newly identified genes act downstream of or in parallel to tub-1, individual mutations are introduced into a tub-1(−) background. Mutations in genes that act in parallel to tub-1 enhance the tub-1(−) lipid accumulation phenotype, while those that act downstream of tub-1 have no effect. Genes that act downstream of tub-1 are cloned using SNP mapping and cosmid rescue. Genes of interest are further analysed, first by GFP/RFP tagging and colocalisation with TUB-1 at a cellular and subcellular level.

Several classes of mutants are expected to be identified in this screen. Of greatest interest are worms that have loss-of-function mutations in genes that act downstream of tub-1. In addition, worms that have loss-of-function mutations in genes that act in parallel to tub-1 are likely to be recovered. tub-1 mutations are known to cause increased fatty acid uptake, but this process is likely to be controlled by multiple pathways. Pathways that synergise with the kat-1(mg368) mutation and cause a dramatic increase in lipid accumulation in C. elegans are identified using this screening strategy. Dominant gain-of-function mutations in genes that regulate fatty acid uptake are also recovered from this screen.

### Functional Genomic Screen Using RNAi Library Feeding

An RNA interference (RNAi) library, covering ˜86% of predicted genes in C. elegans, was used to carry out a functional genomic screen. By feeding worms bacterial clones that generate double-stranded RNA against individual genes, loss of function phenotypes of each gene can be examined. It has been shown that the Nile red staining protocol is compatible with the high throughput format of RNAi screening. Since RNAi usually elicits hypomorphic/weak loss-of-function phenotypes, the proposed RNAi screen allows the identification of genetically lethal loci that could be missed in a forward genetic screen.

The excessive lipid accumulation phenotype of kat-1 tub-1 mutant worms was recapitulated by feeding tub-1(−) worms bacteria expressing double-stranded RNA that is complementary to the kat-1 gene. It was reasoned that RNAi against genes that act downstream of or in parallel to tub-1 in kat-1(−) worms could also elicit an excessive lipid accumulation phenotype. kat-1(−) worms were fed dsRNA that targeted genes on chromosome IV (2693 clones). One clone conferred an excessive lipid accumulation phenotype in kat-1(−) worms, but not in wild-type worms, thus mimicking the synergistic effect of tub-1 on a kat-1 loss of function mutation. C29E6.4 encodes a protein with 5 PAN domains at the N-terminus. The PAN domain is thought to mediate a protein-protein interaction. At least 6 proteins in the worm genome have a similar modular organization as C29E6.4. Four of them have signal peptides. Systematic screening of the entire genome is carried out to identify additional genes that regulate lipid accumulation.

Since RNAi does not work efficiently in the nervous system of C. elegans, and tub-1 is expressed exclusively in neurons, the kat-1(mg368) mutation may be introduced into a eri-1; lin-15B genetic background that has been shown to allow RNAi to function in neurons. This facilitates identification of genes that act both proximally and distally to tub-1. To determine whether these genes act downstream of tub-1, tub-1(−) animals are subjected to RNAi against the candidate genes. The bona fide tub-1 downstream genes are not expected to enhance the lipid accumulation phenotype of tub-1(−) worms. GFP/RFP tagged candidate proteins are expressed in transgenic worms and their colocalisation with TUB-1 at a cellular and subcellular level is examined.

### Analysis of Lipid Profile of kat-1 tub-1 Mutant Worms

Differences in the amount of total lipid extracted from C. elegans correlates with the intensity of the worm's Nile red staining. Because Nile red does not preferentially stain a particular class of lipid molecules, it cannot indicate what class of lipid molecules is being accumulated. Thus, to identify whether the lipids accumulated in kat-1 tub-1 mutant worms are the very long chain fatty acids that are normally metabolized by kat-1(+) dependent β-oxidation, or whether there is a general increase in the levels of many classes of lipid molecules, the lipid profiles of wild-type, together with kat-1, tub-1, and kat-1 tub-1 mutant worms are analysed by gas chromatography/mass spectroscopy. Particular attention is paid to the relative amount of very long chain fatty acids (C22, C24, C26 and C27), since an elevation of very long chain fatty acids is speculated to serve as the primary trigger for a global increase in lipid content. This is an attractive model since kat-1 tub-1 mutant worms show an increase in the uptake and retention of a long chain, fluorescent BODIPY-labeled fatty acid analogue that is not normally a substrate for peroxisomal β-oxidation.

ald-1 Deletion

Since kat-1 is involved in a multi-step peroxisomal β-oxidation pathway, mutations in other components of the same pathway are also likely to result in excessive lipid accumulation in a tub-1(−) background. From the C. elegans Knockout Consortium, a mutant strain having an ald-1 deletion was obtained. ald-1 is the C. elegans homologue of the human ABCD1 gene, which encodes a putative very long chain fatty acids transporter. A mutation in ABCD1 causes an accumulation of very long chain fatty acids and X-linked adrenoleukodystrophy in humans.

kat-2 Deletion

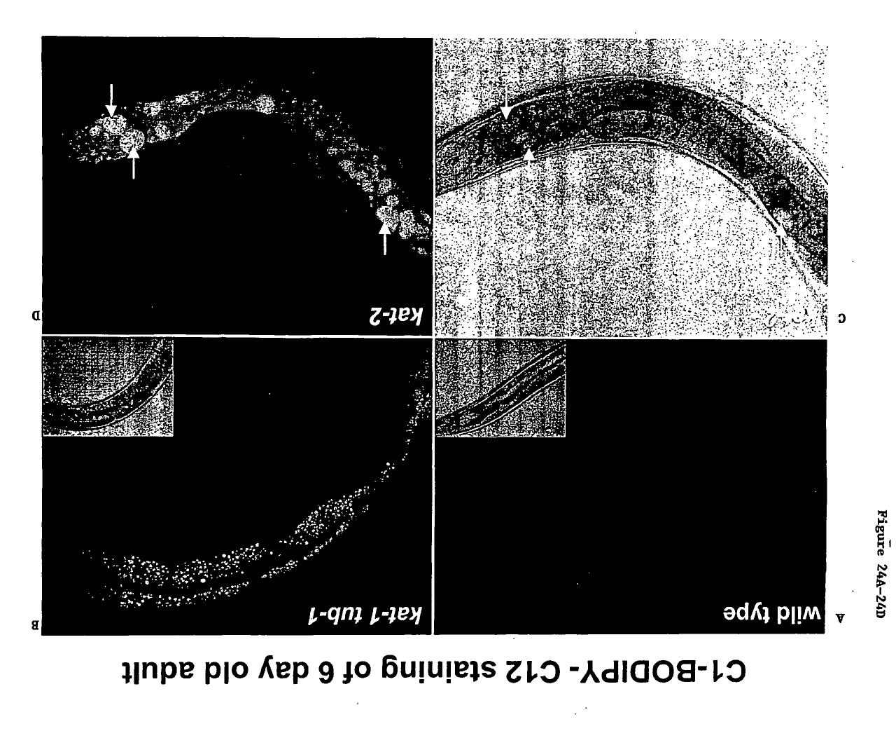

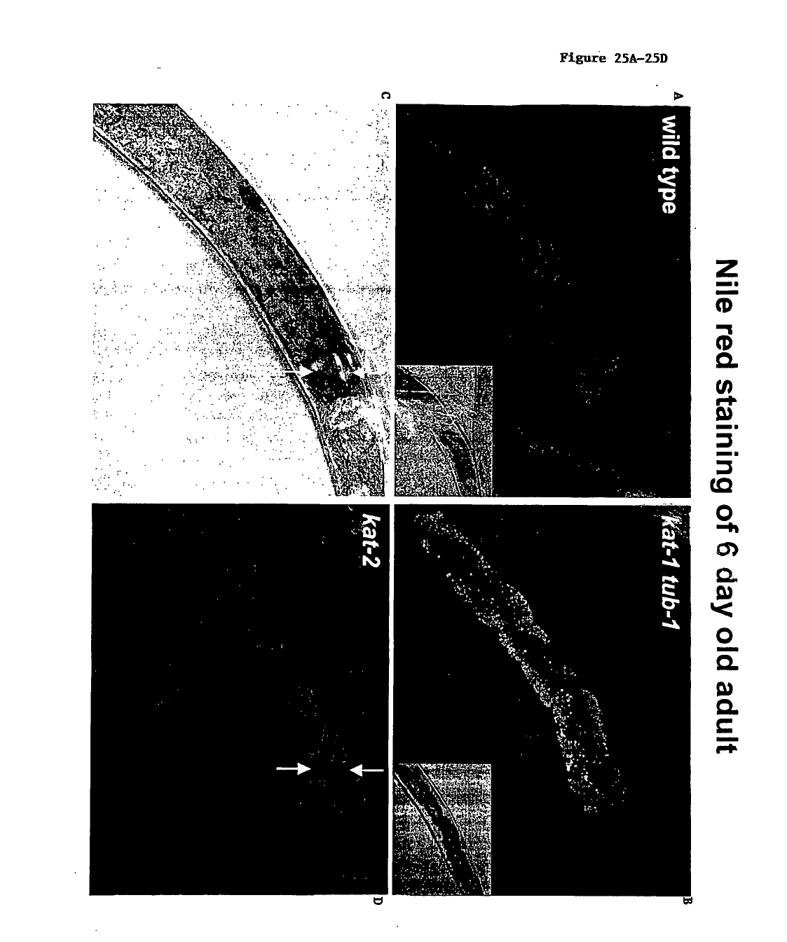

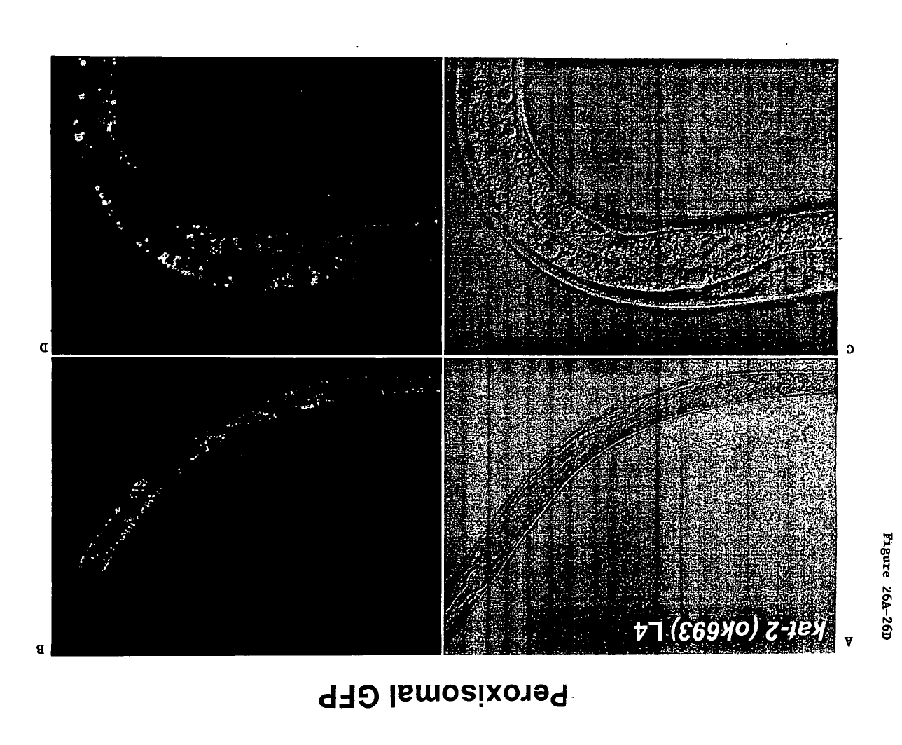

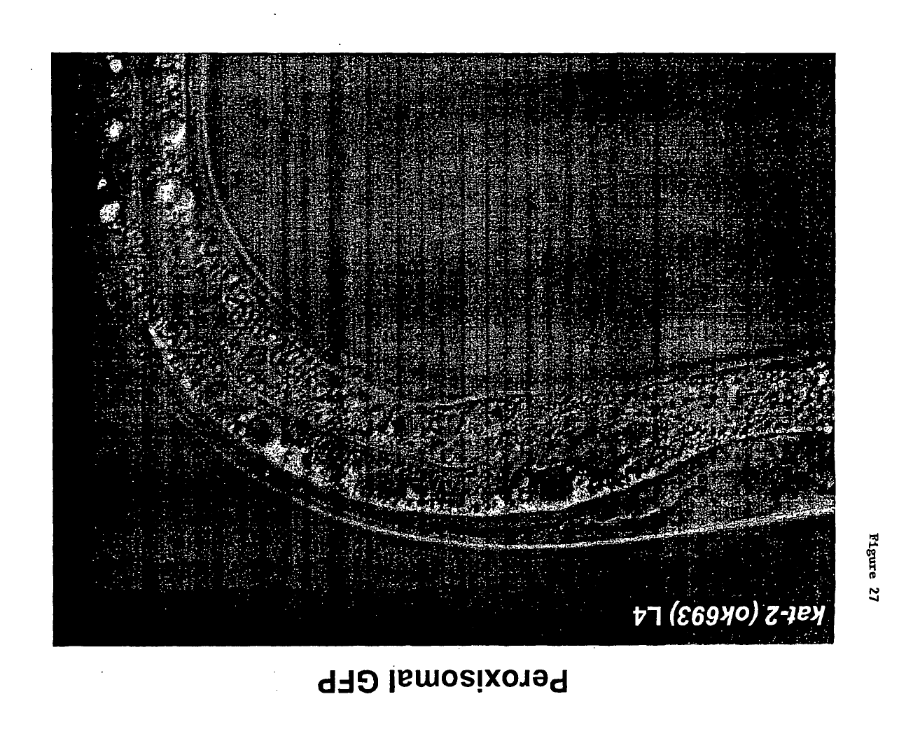

kat-2 is the only other peroxisomal 3-ketoacyl-coA thiolase encoded by the C. elegans genome (Y57A10C.6). A kat-2 deletion was obtained from the C. elegans genome consortium. As shown in FIGS. 24A-24D, giant lipid vesicles, which are stained by C1-Bodipy-C12, are present in kat-2 mutants. Interestingly, these giant lipid vesicles are not stained by Nile Red (FIGS. 25A-24D), which fluoresces in a neutral lipid environment. This may suggest that kat-2 lipid vesicles mutants have a non-neutral pH. kat-2 deletion mutants have altered peroxisome distribution relative to wild-type worms FIGS. 26A-26D and FIG. 27.

tub-1; ald-1 and tub-1 kat-2 double mutant worms are generated and their lipid content examined. Finally, kat-1 kat-2 double mutant worms are generated. This would be the first example of complete peroxisomal β-oxidation blockage in an animal. The viability, reproductive development, and lipid content of these worms is determined.

### Lipid Partitioning

Another object of the invention is to identify mechanisms used for the control of intracellular lipid partitioning. Lipid droplets, which are composed of a hydrophobic core of neutral lipids surrounded by a phospholipid monolayer sprinkled with proteins, such as perilipins, are present in animal and plant cells. The abundance and size of these neutral lipid droplets are dynamic, varying with cellular energy demand and input.

As described above, worms that are defective in peroxisomal branched chain fatty acid β-oxidation are deficient in neutral lipid particles and contain giant membrane bound lipid vesicles with an altered pH. These vesicles may represent enlarged pre-existing lipid droplets or organelles or they may be novel compartments that specifically sequester excess branched chain fatty acids. The inability to break down branched chain fatty acids results in Refsum disease, a progressive neuropathy, in humans. Screening of small molecules and natural products that suppress the biogenesis of abnormal lipid vesicles in C. elegans mutant worms identifies therapeutic compounds and lead compounds for the treatment of fatty acid metabolism disorders, such as Refsum disease.

The experiments described above were carried out using the following experimental designs and methods.

### Experimental Design and Methods

C. elegans Strains

C. elegans strains used were as follows: wild-type N2 Bristol, bbs-1(mg409) I, bbs-1(mg409) I; kat-1(mg368) II, kat-1(mg368) II, tub-1(nr2004) II, tub-1(nr2044) II, kat-1(mg368) tub-1(nr2004) II, che-2(e1033) X, osm-5(p813) X, daf-6(e1377) X, kat-1(mg368) II; che-2(e1033) X, kat-1(mg368) II; osm-5(p813) X, kat-1(mg368) II; daf-6(e1377) X.

mgIs43: Is[ges-1p::GFP-PTS1]. Intestine specific expression of GFP targeted to the peroxisomal matrix.
mgIs48: Is[elt-2p::GFP-mito]. Intestine specific expression of GFP targeted to the mitochondrial matrix.
nrIs8, nrIs9: Is[snb-1p::tub-1 cDNA]
mgEx680: Ex[kat-1p::GFP]
mgEx681, mgEx682: Ex[kat-1p::kat-1 cDNA; mec-7::GFP]
mgEx683, mgEx684: Ex[ges-1p::kat-1 cDNA; mec-7::GFP]
mgEx685, mgEx686: Ex[tub-1p::kat-1 cDNA; mec-7::GFP]
mgEx687: Ex[bbs-1 genomic fragment; mec-7::GFP]
mgEx688: Ex[che-2p::bbs-1 cDNA; ttx-3::dsRed]
mgEx689: Ex[tub-1p::GFP::tub-1]
mgEx690: Ex[ges1p::GFP-PTS1_{KAT-1}]

DNA Constructs

The TUB-1::GFP construct was based on pPD49.78 (provided by A. Fire). PCR products encompassing the tub-1 5′regulatory region (1.4 kb), GFP-coding region from pPD95.75 (0.9 kb) and tub-1 coding region, including all exons and introns (2.2 kb), were subcloned into pPD49.78 via HindIII/XbaI, XbaI/SmaI and NheI/Asp718 restriction sites respectively. The tub-1 coding sequence was verified by sequencing.

For the kat-1p::GFP construct, PCR products that encompass the kat-1 5′ regulatory region (1.5 kb) and the GFP-coding region plus unc-54 3′UTR from pPD95.79 (2 kb) were assembled using a recombinant PCR method. For tissue specific rescue of kat-1, a kat-1 cDNA (provided by Mark. Vidal) was first subcloned into pPD49.78, in order to append the unc-54 3′UTR to its 3′ end. The following promoters were amplified from N2 genomic DNA: kat-1 (1.5 kb), ges-1 (2.5 kb) (Aamodt et al., 1991) and tub-1(1.4 kb). The primer sequences defining the 5′ end of the promoters used are as follows:

kat-1,
5′-acctacgtcgcaagaatgaaac-3′;
ges-1,
5′-ttaacaaggacgatggtccag-3′;
tub-1,
5′-gaaaattccgctaaacttaaac-3′.

Each promoter fragment encompassed sequence immediately 5′ to the start codon of the respective gene. The promoter fragments were then fused to the kat-1 cDNA and unc-54 3′UTR by recombinant PCR.

The consensus peroxisomal targeting sequence (PTS1) (which was described by Motley et al., EMBO Rep 1:40-46, 2000), PLHSKL, was appended to the C-terminus of GFP using a PCR based method and the GFP-PTS1 coding sequence was then re-introduced into the pPD95.79 vector backbone. The C-terminus of KAT-1, GMVIQKL, was similarly appended to the C-terminus of GFP to yield GFP-PTS1_{KAT-1}.

Intestine specific expression was achieved by using the ges-1 promoter. PCR products encompassing the ges-1 promoter, GFP-PTS1 and unc-54 3′UTR were assembled by recombinant PCR.

For expression of mitochondrial matrix targeted GFP in the intestine, the elt-2 promoter (5.1 kb) (Fukushige et al., Dev Biol 198:286-302, 1998) was subcloned into pPD96.32 via XbaI/Asp718 restriction sites.

For ciliated neuron expression of bbs-1::GFP, PCR products of the che-2 promoter (700 bp) (Fujiwara et al., Neuron 36:1091-1102, 1999), bbs-1 cDNA (kindly provided by M. Vidal) and GFP-coding region with the unc-54 3′UTR from pPD95.79 were assembled by recombinant PCR.

Nile Red and C1-BODIPY-C12 Staining

Nile red (Molecular Probes N-1142) was dissolved in acetone to produce a 0.5 mg/ml stock solution, which was maintained at room temperature. C1-BODIPY-C12 (Molecular Probes D-3823) was dissolved in DMSO to produce a 5 mM stock solution, which was maintained at −20° C. Stock solutions were freshly diluted in 1×PBS to 1 μg/ml (Nile red) or 1 μM (C1-BODIPY-C12), and 0.5 ml of the diluted solution was applied to the surface of NGM plates (˜10 ml agar) seeded with E. coli OP50. The seeded plates were allowed to dry in a laminar flow hood and they were equilibrated overnight at room temperature prior to use. For Nile red staining, worms from a synchronous egg lay were allowed to develop on Nile red containing plates. For C1-BODIPY-C12 staining, L4 stage worms were transferred from normal NGM plates onto those containing C1-BODIPY-C12. Similar results were obtained when worms from synchronous egg lay were allowed to develop on C1-BODIPY-C12 containing plates.

tub-1(nr2004) Modifier Screen and Cloning of Kat-1

tub-1 (nr2004) unc-4(e120) worms were mutagenised with ethyl methanesulfonate (EMS) using standard procedures. F2 progeny from mutagenised worms were grown on plates with Nile red and 2-day old adult worms that displayed altered Nile red staining when compared with tub-1(nr2004) unc-4(e120) control were selected. 132,000 haploid genomes were screened and 28 mutants were identified that displayed either enhanced or reduced Nile red staining. 5 mutants, mg368, mg399, mg400, mg401, mg402, that showed enhanced Nile red staining failed to complement each other and therefore defined a single complementation group.

mg368 was mapped using a SNP based mapping strategy by crossing mg368 tub-1(nr2004) unc-4(e120) worms with the Hawaiian C. elegans isolate CB4856. F₂ unc progeny that showed enhanced Nile red staining were selected for SNP analysis. mg368 was found to localize to a 50 kb genomic region on LGII between cosmids C55B7 and F45E12. Cosmids in this region were injected into mg368 tub-1(nr2004) unc-4(e120) worms and one cosmid, T02G5, rescued the enhanced Nile red staining phenotype (3/3 transgenic lines). A PCR fragment encompassing the coding and regulatory regions of T02G5.8 also showed rescuing activity (5/5 transgenic lines). Point mutations in the coding region of T02G5.8 were identified for each of the five mutants. We named T02G5.8 kat-1 for its molecular identity of 3-ketoacyl-coA thiolase.

kat-1(mg368) Modifier Screen and Cloning of bbs-1

kat-1(mg368) unc-4(e120) worms were mutagenised with ethyl methanesulfonate (EMS) using standard procedures. F₂ progeny from mutagenised worms were grown on plates with Nile red and 2-day old adult worms that displayed enhanced Nile red staining when compared with kat-1(mg368) unc-4(e120) control were selected. We screened 79,200 haploid genomes and retrieved 41 mutants that displayed enhanced Nile red staining in a kat-1(mg368) dependent manner. Two mutants displayed an additional dye-filling defective phenotype, one of these was mg409. We mapped mg409 by a SNP-based mapping strategy, as described above, and determined that mg409 was located at the right arm of LGI to the right of cosmid F08A8. We sequenced a number of candidate genes in this genomic region and found a point mutation in the gene bbs-1(Y105E8A.5) that introduced a stop codon in exon 7. Subsequent rescue experiments using PCR fragments encompassing the coding and regulatory region of Y015E8A.5 confirmed that mg409 is a mutant allele of bbs-1 (2/2 transgenic lines).

Microscopy

Nile red and C1-BODIPY-C12 stained worms were examined using a ZIESS AXIOPLAN II microscope (Carl Zeiss, Oberkochen, Germany) equipped with GFP and rhodamine filters. Digital images were captured using a CCD camera (Hamamatsu C4742-95-12ER) (Hamamatsu Corp., Shizuoka, Japan). For comparison of staining intensity between worms of different genotypes, images of the anterior intestine were taken with identical settings and exposure times. For quantification of fluorescence intensity, images of the anterior intestine were collected at 12-bit with the raw pixel values within the linear range of the CCD camera as determined by the ‘automatic exposure’ function (OPENLAB) (Improvision, Lexington, Mass., USA). The fluorescence intensity of each image was calculated by dividing the mean pixel intensity of all Nile red staining lipid droplets by the exposure time (OPENLAB) (Improvision, Lexington, Mass., USA).

Confocal microscopy was performed with a LEICA TCS NT confocal laser spectrophotometer (Leica Microsystems, Wetzlar, Germany) using the FITC/TRITC/TRANS filters (400-750 nm). All images were taken with a 63× objective. For TUB-1::GFP images, the images were projections of 16 optical slices (8 μm depth).

### Mammalian Orthologs

Because key mechanisms of body fat and sterol regulation are conserved between mammals and C. elegans, the powerful genetics and genomics of C. elegans can be exploited for the systematic identification of corresponding mammalian genes that regulate lipid accumulation, their interactions, responses to environmental perturbations, and changes over the lifespan of the worm. Moreover, the comprehensive RNAi system described herein allows for the rapid identification and classification of new fat metabolism regulator genes in C. elegans. Many of these genes have mammalian orthologs not previously associated with fat metabolism. These mammalian genes may be unidentified components of known lipid accumulation pathways, or present new paradigms for metabolism regulation. Given the regulatory interactions that exist between fat cells and the CNS, the study of lipid accumulation in a physiologically intact animal, such as C. elegans, can provide insights unattainable in other model systems, such as cultured mammalian adipocytes.

### Microarrays

The global analysis of gene expression using gene chips can provide insights into gene expression perturbations in tissues associated with obesity. Such studies can compare the expression profiles of mammalian genes of the invention in tissues (e.g., ciliated neurons, hypothalamus, adipocytes) of obese mice or humans relative to non-obese mice or humans. For example, microarrays would allow comparisons to be made between gene expression profiles present in ciliated neurons of individuals with Bardet-Biedl or Refsum syndrome relative to corresponding cells in a control. Genes identified using such methods are likely to function in lipid accumulation, energy intake, or metabolism control. In another example, genes whose expression is modulated in mice having a mutation in murine kat-1 relative to wild-type control mice, represent therapeutic targets for further analysis. The function of such targets may be explored in C. elegans, in cell culture models of fat deposition, and in humans.

Microarrays may be prepared, used, and analyzed using methods known in the art. (See, e.g., Brennan et al., U.S. Pat. No. 5,474,796; Schena et al., Proc. Natl. Acad. Sci. 93:10614, 1996; Baldeschweiler et al., PCT application WO95/251116, 1995; Shalon, D. et al., PCT application WO95/35505, 1995; Heller et al., Proc. Natl. Acad. Sci. 94:2150, 1997; and Heller et al., U.S. Pat. No. 5,605,662; MacBeath et al., Science 289:1760-1763, 2000; De Wildt et al., Nature Biotechnol. 18, 989-994, 2000; Fung et al., Curr. Opin. Biotechnol. 12:65-69, 2001).

siRNA

Short twenty-one to twenty-five nucleotide double stranded RNAs are effective at down-regulating gene expression in mammalian tissue culture cell lines (Elbashir et al., Nature 411:494-498, 2001, hereby incorporated by reference). Using such methods, mammalian orthologs of genes identified herein (e.g., kat-1, egl-4, ald-1, che-2, osm-5, daf-6, tax-2, and tax-4) are inactivated and analyzed for fat phenotype in vitro in tissue culture or in vivo in animal models. The nucleic acid sequence of mammalian gene orthologs can be used to design small interfering RNAs (siRNAs) that will inactivate mammalian genes that regulate lipid accumulation for the treatment of obesity or obesity-related disease.

Given the sequence of a mammalian fat metabolism regulator gene in a pathway that regulates lipid accumulation or in a related pathway, siRNAs may be designed to inactivate that gene. For example, for a gene that consists of 2000 nucleotides, 1,978 different twenty-two nucleotide oligomers could be designed; this assumes that each oligomer has a two base pair 3′ overhang, and that each siRNA is one nucleotide residue from the neighboring siRNA. For RNAi, only a few of these twenty-two nucleotide oligomers would be needed; approximately one dozen siRNAs, evenly spaced across the 2,000 nucleotide gene, could be sufficient to significantly reduce mammalian gene activity. These siRNAs can be transferred into mammalian cells in culture, and the effect of the siRNAs on the cultured cells fat content would then be assayed using Nile Red, such methods are standard in the art and are described by Elbashir et al., (Nature 411:494-498, 2001, hereby incorporated by reference). Alternatively, siRNAs could be injected into an animal, for example, into the bloodstream (McCaffrey et al., Nature 418:38-92002). Thus, based on the mammalian genes identified, oligonucleotides may be designed to inhibit mammalian gene activity. Those siRNAs that are effective in reducing lipid accumulation in cultured cells can be used as therapeutics.

### Druggable Targets

Enzymes with small molecule substrates have been traditional targets for drug development. Examples include kinases, phosphatases, lipases, synthases, ABC transporters, nuclear hormone receptors, small molecule receptors, and small molecule transporters. Many small molecule drugs have already been developed. The chemical backbone of drugs designed against a class of enzymes with small substrate molecules, e.g. kinases or nuclear hormone receptors, may be used as a starting point for developing and designing drug targets against other members within that class of enzymes. The genes described herein function in lipid accumulation. The mammalian orthologs of the genes disclosed herein (e.g., kat-1, kat-2, egl-4, ald-1, che-2, osm-5, daf-6, tax-2, and tax-4) are novel candidates for the development of drugs for the treatment of obesity and obesity-related diseases.

### Transgenic Rodents

Yet another method for assessing the utility of targets, is the use of transgenic rodents that are widely used as mammalian models of obesity. Examples include the following trangenic/mutant mice: ob/ob, db/db, fat/fat, tubby/tubby, -5HTRc/5HTRc, MC3R/MC3R, MC4R/MC4R, BRC3/BRC3, 11-β-HSD-1/11-β-HSD-1, CYP19/CYP19, ADR3b/ADR3b, Ppara-α/Ppara-α, Esr-α/Esr-α, Pomc/Pomc, Fshr/Fshr, and agouti mice (Brockmann et al., Trends in Genetics 18: 367-376, 2002 and Butler et al., Trends in Genetics 17(10):S50-S54, 2001). These mice display hyperphagia and in some cases increased fat deposits. The genes described herein (e.g., kat-1, kat-2, egl-4, ald-1, che-2, osm-5, daf-6, tax-2, and tax-4) can be studied by assaying the fat phenotype of the obese mutant mice having a second mutation in one of these genes.

Alternatively, obese mice, such as: ob/ob, db/db, fat/fat, tubby/tubby, -5HTRc/5HTRc, MC3R/MC3R, MC4R/MC4R, BRC3/BRC3, 11-β-HSD-1/11-β-HSD-1, CYP19/CYP19, ADR3b/ADR3b, Ppara-α/Ppara-α, Esr-α/Esr-α, Pomc/Pomc, Fshr/Fshr, and agouti mice, may be injected with an siRNA (for example, a twenty-one-nucleotide siRNA) corresponding to a C. elegans gene identified herein (e.g., kat-1, kat-2, egl-4, ald-1, che-2, osm-5, daf-6, tax-2, and tax-4).

### Diagnostics

In other embodiments, methods for diagnosing obesity, an obesity-related disease, or a lipid accumulation disorder are also included in the methods of the invention. In one embodiment, antibodies that specifically bind any of the polypeptides described herein may be used for the diagnosis of obesity, an obesity-related disease, or a lipid accumulation disorder by assaying for an alteration in polypeptide expression (e.g., polypeptide localization or polypeptide level). A variety of protocols for measuring such polypeptides, including immunological methods (such as ELISAs and RIAs) and FACS, are known in the art and provide a basis for diagnosing obesity, an obesity-related disease, or a lipid accumulation disorder.

In another embodiment, hybridization techniques may be used to identify mutations in genes that regulate lipid accumulation or may be used to monitor expression levels of these genes (for example, by Northern analysis, (Ausubel et al., supra). Alterations in expression or a mutation in a gene that functions in lipid accumulation is indicative of obesity, an obesity-related disorder, or a lipid accumulation disorder. Hybridization with PCR probes which are capable of detecting polynucleotide sequences, including genomic sequences, encoding a mammalian ortholog of KAT-1, KAT-2, EGL-4, ALD-1, CHE-2, DAF-6, OSM-5, TAX-2, or TAX-4, or closely related molecules, may be used to identify nucleic acid sequences having a mutation. The specificity of the probe, whether it is made from a highly specific region, e.g., the 5′ regulatory region, or from a less specific region, e.g., a conserved motif, and the stringency of the hybridization or amplification (maximal, high, intermediate, or low), will determine whether the probe identifies only naturally occurring sequences encoding KAT-1, KAT-2, EGL-4, ALD-1, CHE-2, DAF-6, OSM-5, TAX-2, or TAX-4 orthologs, allelic variants, or related sequences.

In further embodiments, oligonucleotides or longer fragments derived from any of the polynucleotide sequences described herein may be used as targets in a microarray. The microarray can be used to monitor the expression level of large numbers of genes simultaneously and to identify genetic variants, mutations, and polymorphisms. This information may be used to determine gene function, to understand the genetic basis of a disorder, to diagnose a disorder, and to develop and monitor the activities of therapeutic agents. Microarrays may be prepared, used, and analyzed using methods known in the art. (See, e.g., Brennan et al., U.S. Pat. No. 5,474,796; Schena et al., Proc. Natl. Acad. Sci. 93:10614, 1996; Baldeschweiler et al., PCT application WO95/251116, 1995; Shalon, D. et al., PCT application WO95/35505, 1995; Heller et al., Proc. Natl. Acad. Sci. 94:2150, 1997; and Heller et al., U.S. Pat. No. 5,605,662.)

In yet another approach, mammals may be diagnosed for a propensity to a lipid accumulation disease or disorder by direct analysis of the sequence of a mammalian ortholog of a kat-1, kat-2, egl-4, ald-1, che-2, daf-6, osm-5, tax-2, or tax-4 gene (for example, by sequence or mismatch detection assays). Mutations in the sequence of such genes, relative to a reference sequence, is diagnositic of obesity, an obesity-related disorder, or a lipid-accumulation disorder, or of the propensity to develop such disorders.

### Screening Assays

As discussed above, the genes identified in the tub pathway and related lipid accumulation pathways modulate the regulation of body fat. Based on this discovery, screening assays may be carried out to identify compounds that enhance or inhibit the action of a polypeptide or the expression of a nucleic acid sequence of the invention. The method of screening may involve high-throughput techniques. In addition, these screening techniques may be carried out in cultured cells or in animals (such as nematodes).

Any number of methods are available for carrying out such screening assays. In one working example, candidate compounds are added at varying concentrations to the culture medium of cultured cells expressing one of the nucleic acid sequences of the invention. Gene expression is then measured, for example, by standard Northern blot analysis (Ausubel et al., supra) or RT-PCR, using any appropriate fragment prepared from the nucleic acid molecule as a hybridization probe. The level of gene expression in the presence of the candidate compound is compared to the level measured in a control culture medium lacking the candidate molecule. A compound which promotes an increase in the expression of a nucleic acid sequence disclosed herein or a functional equivalent is considered useful in the invention; such a molecule may be used, for example, as a therapeutic to delay or ameliorate human diseases associated with obesity, an obesity-related disease, or a lipid accumulation disorder. Such cultured cells include nematode cells (for example, C. elegans cells), mammalian (e.g., adipocytes, ciliated neurons), or insect cells.

In another working example, the effect of candidate compounds may be measured at the level of polypeptide production using the same general approach and standard immunological techniques, such as Western blotting or immunoprecipitation with an antibody specific for a polypeptide of the invention. For example, immunoassays may be used to detect or monitor the expression of at least one of the polypeptides of the invention in an organism. Polyclonal or monoclonal antibodies (produced by standard techniques) that are capable of binding to such a polypeptide may be used in any standard immunoassay format (e.g., ELISA, Western blot, or RIA assay) to measure the level of the polypeptide. A compound that promotes an increase in the expression of the polypeptide is considered particularly useful. Again, such a molecule may be used, for example, as a therapeutic to delay or ameliorate human diseases associated with excess body weight, obesity, or lipid accumulation.

In yet another working example, candidate compounds may be screened for those that specifically bind to and agonize or antagonize a polypeptide of the invention. The efficacy of such a candidate compound is dependent upon its ability to interact with a nucleic acid or polypeptide of the invention or a functional equivalent thereof. Such an interaction can be readily assayed using any number of standard binding techniques and functional assays (e.g., those described in Ausubel et al., supra). For example, a candidate compound may be tested in vitro for interaction and binding with a polypeptide of the invention and its ability to modulate body fat metabolism may be assayed by any standard assay (e.g., those described herein).

In one particular working example, a candidate compound that binds to a polypeptide may be identified using a chromatography-based technique. For example, a recombinant polypeptide of the invention may be purified by standard techniques from cells engineered to express the polypeptide (e.g., those described above) and may be immobilized on a column. A solution of candidate compounds is then passed through the column, and a compound specific for the polypeptide is identified on the basis of its ability to bind to the polypeptide and be immobilized on the column. To isolate the compound, the column is washed to remove non-specifically bound molecules, and the compound of interest is then released from the column and collected. Compounds isolated by this method (or any other appropriate method) may, if desired, be further purified (e.g., by high performance liquid chromatography). In addition, these candidate compounds may be tested for their ability to affect fat metabolism (e.g., as described herein). Compounds isolated by this approach may also be used, for example, as therapeutics to delay or ameliorate, human diseases associated with excess body weight or obesity. Compounds that are identified as binding to polypeptides of the invention with an affinity constant less than or equal to 10 mM are considered particularly useful in the invention.

In another example, compounds that modulate the biological activity of a KAT-1, KAT-2, EGL-4, ALD-1, CHE-2, DAF-6, OSM-5, TAX-2, or TAX-4 polypeptide are identified using any assay known in the art or described herein, for example, lipid accumulation assays with Nile Red or C1-BODIPY-C12. Compounds that modulate the biological activity of a KAT-1 or KAT-2 polypeptides are identified by assaying for those that alter peroxisomal 3-ketoacyl-coA thiolase activity, or by assaying for compounds that alter fatty acid metabolism as described by (Seedorf et al., Genes and Development 12: 1189-1201, 1998). Compounds that alter EGL-4 activity are identified by assaying for those that alter cGMP dependent protein kinase activity (Busch et al., JBC 277: 34048-34054, 2002). Compounds that alter ALD-1 biological activity are identified by assaying for those that alter very long chain fatty acid transporter activity. Compounds that alter tax-2 or tax-4 biological activity are identified by assaying for those that alter the activity of a cyclic nucleotide-gated channel (Komatsu et al., Brain Research 821: 160-168, 1999). Compounds that alter che-2, tax-2, or tax-4 biological activity are identified by assaying for those that alter C. elegans ciliated neuron function or chemotaxis. In addition, compounds that alter CHE-2, DAF-6, OSM-5, TAX-2, or TAX-4 biological activity are identified by assaying for those that cause a wild-type C. elegans to phenocopy the defects present in a che-2, daf-6, osm-5, tax-2, or tax-4 mutant worm.

Potential agonists and antagonists include organic molecules, peptides, peptide mimetics, polypeptides, nucleic acids, and antibodies that bind to a nucleic acid sequence or polypeptide of the invention and thereby increase or decrease its activity. Potential agonists also include small molecules that bind to and occupy the binding site of the polypeptide thereby preventing binding to cellular binding molecules, such that normal biological activity is prevented.

Each of the DNA sequences provided herein may also be used in the discovery and development of therapeutic lead compounds. The encoded protein, upon expression, can be used as a target for the screening of fat metabolism regulating drugs. Additionally, the DNA sequences encoding the amino terminal regions of the encoded protein or Shine-Delgarno or other translation facilitating sequences of the respective mRNA can be used to construct antisense sequences to control the expression of the coding sequence of interest. Such sequences may be isolated by standard techniques (Ausubel et al., supra).

The antagonists and agonists of the invention may be employed, for instance, to delay or ameliorate human diseases associated with obesity, an obesity-related disease (e.g., Bardet-Biedl syndrome), or a lipid accumulation disorder (e.g., Refsum disease).

Optionally, compounds identified in any of the above-described assays may be confirmed as useful in delaying or ameliorating human diseases associated with excess body weight, an obesity-related disease (e.g., Bardet-Biedl syndrome), or a lipid accumulation disorder (e.g., Refsum disease), in either standard tissue culture methods (e.g. Nile Red staining of fat storage in cultured cells) or animal models of obesity (e.g., naturally occurring rodent mutants, such as, for example, Ob (leptin), db (leptin receptor), fat-1 (carboxypeptidase E), 5-HTR (serotonin receptor, and tubby) and, if successful, may be used as therapeutics for the treatment of obesity or disorders related to fat metabolism.

Small molecules of the invention preferably have a molecular weight below 2,000 daltons, more preferably between 300 and 1,000 daltons, and most preferably between 400 and 700 daltons. It is preferred that these small molecules are organic molecules.

### Test Compounds and Extracts

In general, compounds capable of delaying or ameliorating human diseases associated with obesity, an obesity-related disease, or a fat metabolism disorder are identified from large libraries of both natural product or synthetic (or semi-synthetic) extracts or chemical libraries according to methods known in the art. Those skilled in the field of drug discovery and development will understand that the precise source of test extracts or compounds is not critical to the screening procedure(s) of the invention. Compounds used in screens may include known compounds (for example, known therapeutics used for other diseases or disorders). Alternatively, virtually any number of unknown chemical extracts or compounds can be screened using the methods described herein. Examples of such extracts or compounds include, but are not limited to, plant-, fungal-, prokaryotic- or animal-based extracts, fermentation broths, and synthetic compounds, as well as modification of existing compounds. Numerous methods are also available for generating random or directed synthesis (e.g., semi-synthesis or total synthesis) of any number of chemical compounds, including, but not limited to, saccharide-, lipid-, peptide-, and nucleic acid-based compounds. Synthetic compound libraries are commercially available from Brandon Associates (Merrimack, N.H.) and Aldrich Chemical (Milwaukee, Wis.). Alternatively, libraries of natural compounds in the form of bacterial, fungal, plant, and animal extracts are commercially available from a number of sources. In addition, natural and synthetically produced libraries are produced, if desired, according to methods known in the art, e.g., by standard extraction and fractionation methods. Furthermore, if desired, any library or compound is readily modified using standard chemical, physical, or biochemical methods.

In addition, those skilled in the art of drug discovery and development readily understand that methods for dereplication (e.g., taxonomic dereplication, biological dereplication, and chemical dereplication, or any combination thereof) or the elimination of replicates or repeats of materials already known for their fat metabolism regulating activity should be employed whenever possible.

When a crude extract is found to lipid accumulation, or to bind to a protein that regulates lipid accumulation activity, further fractionation of the positive lead extract is necessary to isolate chemical constituents responsible for the observed effect. Thus, the goal of the extraction, fractionation, and purification process is the careful characterization and identification of a chemical entity within the crude extract having fat metabolism regulating activity. Methods of fractionation and purification of such heterogenous extracts are known in the art. If desired, compounds shown to be useful agents to delay or ameliorate an obesity-related disease (e.g., Bardet-Biedl syndrome) or a lipid accumulation disorder (e.g., Refsum disease) are chemically modified according to methods known in the art.

All publications and references, including but not limited to patents and patent applications, cited in this specification are herein incorporated by reference in their entirety as if each individual publication or reference were specifically and individually indicated to be incorporated by reference herein as being fully set forth. Any patent application to which this application claims priority is also incorporated by reference herein in its entirety in the manner described above for publications and references.

What is claimed is:

1. A method for identifying a nucleic acid molecule encoding a polypeptide that regulates lipid accumulation, said method comprising:
(a) providing a mutagenized nematode having a pre-existing mutation in a nucleic acid molecule selected from the group consisting of kat-1, kat-2, bbs-1, egl-4, ald-1, che-2l, daf-6, C29E6.4, osm-5, tax-2, and tax-4;
(b) contacting said nematode with a dye that stains body fat; and
(c) comparing the body fat staining of said mutagenized nematode to a control nematode, wherein a mutation in a nucleic acid molecule encoding a polypeptide that regulates lipid accumulation is identified by an alteration in body fat staining.

2. A method for identifying a nucleic acid molecule that encodes a polypeptide that regulates lipid accumulation, said method comprising:
(a) contacting a nematode having a pre-existing mutation in a nucleic acid molecule selected from the group consisting of kat-1, kat-2, bbs-1, egl-4, ald-1, che-2, daf-6, C29E6.4, osm-5, tax-2, and tax-4 with a candidate inhibitory nucleic acid;
(b) contacting said nematode with a dye that stains body fat; and
(c) comparing the body fat staining of said nematode contacted with said inhibitory nucleic acid molecule to a control nematode, wherein an alteration in body fat staining identifies the sense nucleic acid molecule corresponding to said inhibitory nucleic acid molecule as a nucleic acid molecule encoding a polypeptide that regulates lipid accumulation.

3. A method for identifying a candidate compound that modulates lipid accumulation, said method comprising:
(a) providing a cell expressing at least one nucleic acid molecule selected from the group consisting of kat-1, kat-2, egl-4, ald-1, che-2, daf-6, osm-5, C29E6.4, tax-2 and tax-4;
(b) contacting said cell with a candidate compound; and
(c) comparing the expression of said nucleic acid molecule in said cell contacted with said candidate compound with the expression of said nucleic acid molecule in a control cell, wherein an alteration in said expression identifies said candidate compound as a candidate compound that modulates lipid accumulation.

4. The method of claim 3, wherein said cell is a nematode cell.

5. The method of claim 3, wherein said cell is a mammalian cell.

6. The method of claim 3, wherein said cell comprises at least two nucleic acid molecules comprising mutations.

7. A method for identifying a candidate compound that modulates lipid accumulation, said method comprising:
(a) providing a nematode cell expressing at least one nucleic acid molecule selected from the group consisting of kat-1, kat-2, bbs-1, egl-4, ald-1, che-2, daf-6, osm-5, C29E6.4, tax-2, and tax-4;
(b) contacting said cell with a candidate compound; and
(c) comparing the expression of said nucleic acid molecule in said cell contacted with said candidate compound with the expression of said nucleic acid molecule in a control cell, wherein an alteration in said expression identifies said candidate compound as a candidate compound that modulates lipid accumulation.

8. The method of claim 7, wherein said screening method identifies a compound that increases or decreases transcription of said nucleic acid molecule.

9. The method of claim 7, wherein said screening method identifies a compound that increases or decreases translation of an mRNA transcribed from said nucleic acid molecule.

10. The method of claim 7, wherein said cell is in a nematode.

11. The method of claim 7, wherein said cell comprises at least two nucleic acid molecules comprising mutations.

12. A method for identifying a candidate compound that regulates lipid accumulation, said method comprising:
(a) providing a cell expressing a polypeptide selected from the group consisting of KAT-1, KAT-2, EGL-4, ALD-1, CHE-2, DAF-6, OSM-5, C29E6.4, TAX-2, and TAX-4;
(b) contacting said cell with a candidate compound; and
(c) comparing the biological activity of said polypeptide in said cell contacted with said candidate compound to a control cell, wherein an alteration in said biological activity of said polypeptide identifies said candidate compound as a candidate compound that modulates lipid accumulation.

13. The method of claim 12, wherein said cell is a nematode cell.

14. The method of claim 13, wherein said nematode cell is in a nematode.

15. The method of claim 12, wherein said cell is a mammalian cell.

16. The method of claim 12, wherein said biological activity is monitored with an enzymatic assay.

17. The method of claim 12, wherein said biological activity is monitored with an immunological assay.

18. The method of claim 12, wherein said biological activity is monitored by detecting fat levels.

19. A method for identifying a candidate compound that modulates lipid accumulation, said method comprising the steps of a) contacting a polypeptide selected from the group consisting of KAT-1, KAT-2, EGL-4, ALD-1, CHE-2, DAF-6, OSM-5, C29E6.4, TAX-2, and TAX-4, or a fragment thereof, or an ortholog thereof, with a candidate compound; and
b) detecting binding of said candidate compound to said polypeptide, wherein said binding identifies the candidate compound as a compound that modulates lipid accumulation.

20. A transgenic organism overexpressing a kat-1, kat-2, egl-4 gain-of-function, bbs-1, C29E6.4, or ald-1 nucleic acid molecule, or fragment thereof, wherein expression of the protein product encoded by said nucleic acid molecule alters lipid accumulation in said organism.

21. The transgenic organism of claim 20, wherein said organism is a nematode.

22. The transgenic organism of claim 20, wherein said organism is a rodent and said polypeptide is an ortholog of KAT-1, KAT-2, C29E6.4, or EGL-4 gain-of-function.

23. A nematode comprising a mutation in a nucleic acid sequence selected from the group consisting of kat-1, kat-2, egl-4 gain-of-function, ald-1, C29E6.4, and bbs-1.

24. A mammal comprising at least one mutation in a nucleic acid sequence selected from the group consisting of kat-1, che-2, daf-6, osm-5, C29E6.4, tax-2, tax-4 or egl-4 gain-of-function.

25. A double-stranded RNA corresponding to at least a portion of a nucleic acid molecule selected from the group consisting of kat-1, kat-2, egl-4, ald-1, che-2, daf-6, osm-5, C29E6.4, tax-2, and tax-4, or an ortholog thereof, wherein said double-stranded RNA when introduced to a cell is capable of altering the level of lipid accumulation in said cell.

26. An antisense nucleic acid molecule complementary to at least a portion of a nucleic acid molecule selected from the group consisting of kat-1, kat-2, egl-4, ald-1, che-2, daf-6, osm-5, C29E6.4, tax-2, and tax-4, or an ortholog thereof, wherein said antisense nucleic acid molecule when introduced to a cell is capable of decreasing expression from the nucleic acid molecule to which it is complementary.

27. A method for diagnosing an organism having, or having a propensity to develop a lipid accumulation disorder, obesity, or an obesity-related disease, said method comprising detecting an alteration in the sequence of a nucleic acid molecule selected from the group consisting of kat-1, kat-2, C29E6.4, egl-4, ald-1, che-2, daf-6, osm-5, tax-2, and tax-4, or an ortholog thereof.

28. A method for diagnosing an organism having, or having a propensity to develop, a lipid accumulation disorder, obesity, or an obesity-related disease, said method comprising detecting an alteration in the expression of a nucleic acid molecule selected from the group consisting of kat-1, kat-2, egl-4, ald-1, che-2, daf-6, osm-5, C29E6.4, tax-2, and tax-4, or an ortholog thereof, relative to the wild-type level of expression.

29. A method for diagnosing an organism having, or having a propensity to develop, a lipid accumulation disorder, obesity, or an obesity-related disease, said method comprising detecting an alteration in the biological activity of a polypeptide selected from the group consisting of KAT-1, KAT-2, EGL-4, ALD-1, CHE-2, DAF-6, OSM-5, C29E6.4, TAX-2, and TAX-4, or an ortholog thereof, relative to the wild-type level of activity.

30. A method for modulating lipid accumulation in an organism, said method comprising contacting said organism with an antisense nucleic acid molecule that complements a portion of a nucleic acid molecule selected from the group consisting of kat-1, kat-2, egl-4, ald-1, che-2, daf-6, osm-5, C29E6.4, tax-2, and tax-4, or an ortholog thereof.

31. A method for modulating lipid accumulation in an organism, said method comprising contacting said organism with a dsRNA nucleic acid molecule that corresponds to at least a portion of a nucleic acid molecule selected from the group consisting of kat-1, kat-2, egl-4, ald-1, che-2, daf-6, osm-5, C29E6.4, tax-2, and tax-4, or an ortholog thereof.

32. A method for modulating lipid accumulation in an organism, said method comprising contacting said organism with a nucleic acid molecule selected from the group consisting of kat-1, kat-2, egl-4, ald-1, che-2, daf-6, osm-5, C29E6.4, tax-2, and tax-4, or an ortholog thereof.

33. The method of claim 30, 31, or 32, wherein said organism is a mammal.

34. The method of claim 33, wherein said mammal is a human.

35. A pharmaceutical composition comprising a polypeptide, or portion thereof, selected from the group consisting of mammalian orthologs of KAT-1, KAT-2, EGL-4, ALD-1, CHE-2, DAF-6, OSM-5, C29E6.4, TAX-2, and TAX-4.

36. A pharmaceutical composition comprising a nucleic acid molecule or portion thereof, selected from the group consisting of mammalian orthologs of kat-1, kat-2, egl-4, ald-1, che-2, daf-6, osm-5, C29E6.4, tax-2, and tax-4.


SEQUENCE LISTING (34 sequences)

SEQ ID NO: 1 - DNA, 22 bp, ARTIFICIAL SEQUENCE
  synthetic
acctacgtcgcaagaatgaaac

SEQ ID NO: 2 - DNA, 21 bp, ARTIFICIAL SEQUENCE
  synthetic
ttaacaaggacgatggtccag

SEQ ID NO: 3 - DNA, 22 bp, ARTIFICIAL SEQUENCE
  synthetic
gaaaattccgctaaacttaaac

SEQ ID NO: 4 - protein, 175 aa, Caenorhabditis elegans
SFRSSLSSVTAPELASVAIKAALERGAVKPSSIQEVFLGQVCQANAGQAPARQAALGAGLDLSVAVTTVNKVCSSGLKAIILAAQQIQTGHQDFAIGGGMESMSQVPFYVQRGEIPYGGFQVIDGIVKDGLTDAYDKVHMGNCGEKTSKEMGITRKDQDEYAINSYKKSAKAVEN

SEQ ID NO: 5 - protein, 173 aa, Saccharomyces cerevisiae
GFKGAFKDVNTDYLLYNFLNEFIGRFPEPLRADLNLIEEVACGNVLNVGAGATEHRAACLASGIPYSTPFVALNRQCSSGLTAVNDIANKIKVGQIDIGLALGVESMTNNYKNVNPLGMISSEELQKNREAKKCLIPMGITNENVAANFKISRKDQDEFAANSYQKAYKAKNE

SEQ ID NO: 6 - protein, 167 aa, Yarrowia lipolytica
GGKGLFKDTSSSELLASLLEGLVKESKIDPKLIGDVVCGNVLAAGAGATEHRAACLVAGIPETVPFVALNRQCSSGLMAVNDVANKIRAGQIDIGIGCGVESMSNQYGPNSVTPFSNKFQNNEEAKKCLIPMGITSENVAAKYNVSRKAQDAFAAKSYEKAAAAQAA

SEQ ID NO: 7 - protein, 169 aa, Arabidopsis thaliana
ARRGGFKDTLPDDLLASVLKAVVERTSLDPSEVGDIVVGTVIAPGSQRAMECRVAAYFAGFPDSVPVRTVNRQCSSGLQAVADVAASIRAGYYDIGIGAGVESMSTDHIPGGGFHGSNPRAQDFPKARDCLLPMGITSENVAERFGVTREEQDMAAVESHKRAAAAIAS

SEQ ID NO: 8 - protein, 175 aa, Drosophila melanogaster
SFQSQLAPLTATQLGARAIEAAIEKAGIAKTDVQEVIMGNVVSAGLGQAPARQAAIFAGLPTNVCCTTVNKVCSSGMKAVMLGAQSLMLGYADVVVAGGMESMSNVPYYLKRGATPYGGVNLTDGIVFDGLWDVYNKFHMGNCAENTAKKLEITRQQQDDFAIESYKRSAAAWAN

SEQ ID NO: 9 - protein, 166 aa, Rattus norvegicus
AGRGGFKDTTPDELLSAVLTAVLQDVKLKPECLGDISVGNVLEPGAGAVMARIAQFLSGIPETVPLSAVNRQCSSGLQAVANIAGGIRNGSYDIGMACGVESMSLSNRGNPGNISSRLLESDKARDCLIPMGITSENVAERFGISRQKQDAFALASQQKAASAQSK

SEQ ID NO: 10 - protein, 166 aa, Mus musculus
ASRGGFKNTTPDELLSAVLTAVLQDVRLKPEQLGDISVGNVLEPGAGAVMARIAQFLSGIPETVPLSTVNRQCSSGLQAVANIAGGIRNGSYDIGMACGVESMSLSGMGNPGNISSRLLESEKARDCLTPMGMTSENVAERFGISRQKQDDFALASQQKAASAQSR

SEQ ID NO: 11 - protein, 166 aa, Homo sapiens
AGRGGFKDTTPDELLSAVMTAVLKDVNLRPEQLGDICVGNVLQPGAGAIMARIAQFLSDIPETVPLSTVNRQCSSGLQAVASIAGGIRNGSYDIGMACGVESMSLADRGNPGNITSRLMEKEKARDCLIPMGITSENVAERFGISREKQDTFALASQQKAARAQSK

SEQ ID NO: 12 - protein, 145 aa, Caenorhabditis elegans
ASTLNDGAAAVIVASQEAVSEQSLKPLARILAYGDAATHPLDFAVAPTLMFPKILERAGVKQSDVAQWEVNEAFSCVPLAFIKKLGVDPSLVNPHGGAVSIGHPIGMSGARLITHLVHTLKSGQIGVAAICNGGGGSSGMVIQKL

SEQ ID NO: 13 - protein, 145 aa, Saccharomyces cerevisiae
ASQVSDGVAGVLLARRSVANQLNLPVLGRYIDFQTVGVPPEIMGVGPAYAIPKVLEATGLQVQDIDIFEINEAFAAQALYCIHKLGIDLNKVNPRGGAIALGHPLGCTGARQVATILRELKKDQIGVVSMCIGTGMGAAAIFIKE

SEQ ID NO: 14 - protein, 147 aa, Yarrowia lipolytica
ASQISDGAGAVLLMRRSVAEKLGQPILAKFVHCKTVGVPPELMGIGPAYAIPAVLEDLGLTVNDVDVFEINEAFASQALFSIQHCGIDESKVNPRGGAIAIGHPLGATGARQFATLLSELKESGKKVGVTSMCIGTGMGAASLVVAE

SEQ ID NO: 15 - protein, 166 aa, Arabidopsis thaliana
ASQISDGAGAVLLMKRSLAMKKGLPILGVFRSFAVTGVEPSVMGIGPAVAIPAATKLAGLNVSDIDLFEINEAFASQYVYSCKKLELDMEKVNVNGGAIAIGHPLGATGARCVATLLHEMKRRGKDCRFGVISMCIGTGMGAAAVFERGDSVDNLSNARVANGDSH

SEQ ID NO: 16 - protein, 145 aa, Drosophila melanogaster
ASTLNDGGAAVVLMSAEAAQKAGIKPLARIVAFQDAETDPIDFPIAPALAIPKLLKRAGVRKEDVAMWEVNEAFSLVVLANIKKLDVDPAKVNVHGGAVSIGHPIGMSGARLVAHLSHSLKKGELGCASICNGGGGASSILIEKL

SEQ ID NO: 17 - protein, 150 aa, Rattus norvegicus
SSQVSDGAAAVLLARRSKAEELGLPILGVLRSYAVVGVPPDIMGIGPAYAIPAALQKAGLTVNDIDIFEINEAFASQALYCVEKLGIPAEKVNPLGGAIALGHPLGCTGARQVVTLLNELKRRGTRAYGVVSMCIGTGMGAAAVFEYPGN

SEQ ID NO: 18 - protein, 150 aa, Mus musculus
SSQVSDGAAAVLLARRSKAEELGLPILGVLRSYAVVGVPPDVMGIGPAYAIPAALQKAGLTVNDIDIFEINEAFASQAVYCVEKLGIPAEKVNPLGGAIALGHPLGCTGARQVVTLLNELKRRGRRAYGVVSMCIGTGMGAAAVFEYPGN

SEQ ID NO: 19 - protein, 150 aa, Homo sapiens
SSQVSDGAAAILLARRSKAEELGLPILGVLRSYAVVGVPPDIMGIGPAYAIPVALQKAGLTVSDVDIFEINEAFASQAAYCVEKLRLPPEKVNPLGGAVALGHPLGCTGARQVITLLNELKRRGKRAYGVVSMCIGTGMGAAAVFEYPGN

SEQ ID NO: 20 - protein, 407 aa, Caenorhabditis elegans
MLSSSGHAIRRGITTSAALSNKHAFIVGAARTPIGSFRSSLSSVTAPELASVAIKAALERGAVKPSSIQEVFLGQVCQANAGQAPARQAALGAGLDLSVAVTTVNKVCSSGLKAIILAAQQIQTGHQDFAIGGGMESMSQVPFYVQRGEIPYGGFQVIDGIVKDGLTDAYDKVHMGNCGEKTSKEMGITRKDQDEYAINSYKKSAKAWENGNIGPEVVPVNVKSKKGVTIVDKDEEFTKVNFDKFTSLRTVFQKDGTITAANASTLNDGAAAVIVASQEAVSEQSLKPLARILAYGDAATHPLDFAVAPTLMFPKILERAGVKQSDVAQWEVNEAFSCVPLAFIKKLGVDPSLVNPHGGAVSIGHPIGMSGARLITHLVHTLKSGQIGVAAICNGGGGSSGMVIQKL

SEQ ID NO: 21 - protein, 417 aa, Saccharomyces cerevisiae
MSQRLQSIKDHLVESAMGKGESKRKNSLLEKRPEDVVIVAANRSAIGKGFKGAFKDVNTDYLLYNFLNEFIGRFPEPLRADLNLIEEVACGNVLNVGAGATEHRAACLASGIPYSTPFVALNRQCSSGLTAVNDIANKIKVGQIDIGLALGVESMTNNYKNVNPLGMISSEELQKNREAKKCLIPMGITNENVAANFKISRKDQDEFAANSYQKAYKAKNEGLFEDEILPIKLPDGSICQSDEGPRPNVTAESLSSIRPAFIKDRGTTTAGNASQVSDGVAGVLLARRSVANQLNLPVLGRYIDFQTVGVPPEIMGVGPAYAIPKVLEATGLQVQDIDIFEINEAFAAQALYCIHKLGIDLNKVNPRGGAIALGHPLGCTGARQVATILRELKKDQIGVVSMCIGTGMGAAAIFIKE

SEQ ID NO: 22 - protein, 424 aa, Homo sapiens
MQRLQVVLGHLRGPADSGWMPQAAPCLSGAPQASAADVVVVHGRRTAICRAGRGGFKDTTPDELLSAVMTAVLKDVNLRPEQLGDICVGNVLQPGAGAIMARIAQFLSDIPETVPLSTVNRQCSSGLQAVASIAGGIRNGSYDIGMACGVESMSLADRGNPGNITSRLMEKEKARDCLIPMGITSENVAERFGISREKQDTFALASQQKAARAQSKGCFQAEIVPVTTTVHDDKGTKRSITVTQDEGIRPSTTMEGLAKLKPAFKKDGSTTAGNSSQVSDGAAAILLARRSKAEELGLPILGVLRSYAVVGVPPDIMGIGPAYAIPVALQKAGLTVSDVDIFEINEAFASQAAYCVEKLRLPPEKVNPLGGAVALGHPLGCTGARQVITLLNELKRRGKRAYGVVSMCIGTGMGAAAVFEYPGN

SEQ ID NO: 23 - protein, 593 aa, Homo sapiens
MAAASSSDSDACGAESNEANSKWLDAHYDPMANIHTFSACLALADLHGDGEYKLVVGDLGPGGQQPRLKVLKGPLVMTESPLPALPAAAATFLMEQHEPRTPALALASGPCVYVYKNLRPYFKFSLPQLPPNPLEQDLWNQAKEDRIDPLTLKEMLESIRETAEEPLSIQSLRFLQLELSEMEAFVNQHKSNSIKRQTVITTMTTLKKNLADEDAVSCLVLGTENKELLVLDPEAFTILAKMSLPSVPVFLEVSGQFDVEFRLAAACRNGNIYILRRDSKHPKYCIELSAQPVGLIRVHKVLVVGSTQDSLHGFTHKGKKLWTVQMPAAILTMNLLEQHSRGLQAVMAGLANGEVRIYRDKALLNVIHTPDAVTSLCFGRYGREDNTLIMTTRGGGLIIKILKRTAVFVEGGSEVGPPPAQAMKLNVPRKTRLYVDQTLREREAGTAMHRAFQTDLYLLRLRAARAYLQALESSLSPLSTTAREPLKLHAVVQGLGPTFKLTLHLQNTSTTRPVLGLLVCFLYNEALYSLPRAFFKVPLLVPGLNYPLETFVESLSNKGISDIIKVLVLREGQSAPLLSAHVNMPGSEGLAAA

SEQ ID NO: 24 - protein, 576 aa, Caenorhabditis elegans
MAKPVNVNQSKWTVPVLLKECEIYCPSTCVAFGPILSDNDSKLIIAHGGHRGVNMKLKVFQQLEQLSESSLADMPTALVHFINDLSSIPSIAVAAGPSLLIYKNLKPFYKFTVPSSAINPTESEAWKAVVNKKINGDTLLTVLKRLEDDVAFSKLTPISQTYLRADKETQVVLVEHYGTKIANSATITCIAKLTKSTAEPLDILVIGTEHCEIFLIDSQAFTILETIKIGSVPVNICAYGTYDVDYRLFVQTRASLIFCMKRGEADYQPIVISQSMITSMVLVNKMIVYTTVENLIHFASFRGKKMNTVKCPSKIKMLEPFIYPLKQLAAVIAVFDKEIRMYNEHYLLDTVQYEKPLAWVKYGCYGREDSTLVVAFKDGSIAIQIFRRKANFDTKLDYNQVPQAHALKLQIPKKTKVFIDLTQREVQLGNRIHKVYQKNLFDVKYRLAASYLELTSSASATVSTTTVLPVEISVDIHGFGPTFRMTIHLLSSSKQNLYDMHLSIISDPELYDFDTPLIPVHLLASGQSYSFTTLLYCKDPEKAANCDVRALLVHAKRATPIVTAVIKMPFSEFPLD

SEQ ID NO: 25 - protein, 5 aa, ARTIFICIAL SEQUENCE
  synthetic
KKIAV

SEQ ID NO: 26 - protein, 5 aa, ARTIFICIAL SEQUENCE
  synthetic
KNIAV

SEQ ID NO: 27 - DNA, 15 bp, ARTIFICIAL SEQUENCE
  synthetic
aaaaagattgccgtt

SEQ ID NO: 28 - DNA, 15 bp, ARTIFICIAL SEQUENCE
  synthetic
aaaaatattgccgtt

SEQ ID NO: 29 - protein, 5 aa, ARTIFICIAL SEQUENCE
  synthetic
XXXXX

SEQ ID NO: 30 - protein, 5 aa, ARTIFICIAL SEQUENCE
  synthetic
XXXAX

SEQ ID NO: 31 - protein, 5 aa, ARTIFICIAL SEQUENCE
  synthetic
KKIAV

SEQ ID NO: 32 - protein, 5 aa, ARTIFICIAL SEQUENCE
  synthetic
KNIAV

SEQ ID NO: 33 - protein, 686 aa, HOMO SAPIENS
MGTLRDLQYALQEKIEELRQRDALIDELELELDQKDELIQKLQNELDKYRSVIRPATQQAQKQSASTLQGEPRTKRQAISAEPTAFDIQDLSHVTLPFYPKSPQSKDLIKEAILDNDFMKNLELSQIQEIVDCMYPVEYGKDSCIIKEGDVGSLVYVMEDGKVEVTKEGVKLCTMGPGKVFGELAILYNCTRTATVKTLVNVKLWAIDRQCFQTIMMRTGLIKHTEYMEFLKSVPTFQSLPEEILSKLADVLEETHYENGEYIIRQGARGDTFFIISKGTVNVTREDSPSEDPVFLRTLGKGDWFGEKALQGEDVRTANVIAAEAVTCLVIDRDSFKHLIGGLDDVSNKAYEDAEAKAKYEAEAAFFANLKLSDFNIIDTLGVGGFGRVELVQLKSEESKTFAMKILKKRHIVDTRQQEHIRSEKQIMQGAHSDFIVRLYRTFKDSKYLYMLMEACLGGELWTILRDRGSFEDSTTRFYTACVVEAFAYLHSKGIIYRDLKPENLILDHRGYAKLVDFGFAKKIGFGKKTWTFCGTPEYVAPEIILNKGHDISADYWSLGILMYELLTGSPPFSGPDPMKTYNIILRGIDMIEFPKKIAKNAANLIKKLCRDNPSERLGNLKNGVKDIQKHKWFEGFNWEGLRKGTLTPPIIPSVASPTDTSNFDSFPEDNDEPPPDDNSGWDIDF

SEQ ID NO: 34 - protein, 737 aa, Caenorhabditis elegans
MKQQPPRIYVQVGTRTFEAHELQKLIPQLEEAISRKDAQLRQQQTIVEGHIKRISELEGEVTTLQRECDKLRSVLEQKAQSAASPGGQPPSPSPRTDQLGNDLQQKAVLPADGVQRAKKIAVSAEPTNFENKPATLQHYNKTVGAKQMIRDAVQKNDFLKQLAKEQIIELVNCMYEMRARAGQWVIQEGEPGDRLFVVAEGELQVSREGALLGKMRAGTVMGELAILYNCTRTASVQALTDVQLWVLDRSVFQMITQRLGMERHSQLMNFLTKVSIFQNLSEDRISKMADVMDQDYYDGGHYIIRQGEKGDAFFVINSGQVKVTQQIEGETEPREIRVLNQGDFFGERALLGEEVRTANIIAQAPGVEVLTLDRESFGKLIGDLESLKKDYGDKERLAQVVREPPSPVKIVDDFREEFAQVTLKNVKRLATLGVGGFGRVELVCVNGDKAKTFALKALKKKHIVDTRQQEHIFAERNIMMETSTDWIVKLYKTFRDQKFVYMLLEVCLGGELWTTLRDRGHFDDYTARFYVACVLEGLEYLHRKNIVYRDLKPENCLLANTGYLKLVDFGFAKKLASGRKTWTFCGTPEYVSPEIILNKGHDQAADYWALGIYICELMLGRPPFQASDPMKTYTLILKGVDALEIPNRRIGKTATALVKKLCRDNPGERLGSGSGGVNDIRKHRWFMGFDWEGLRSRTLKPPILPKVSNPADVTNFDNYPPDNDVPPDEFSGWDEGF